(19) United States
(12) Patent Application Publication
(10) Pub. No.: US 2024/0238033 A1
(43) Pub. Date: Jul. 18, 2024

(54) Electrosurgical System

(71) Applicant: Eric R. Cosman, JR., Gloucester, MA (US)

(72) Inventor: Eric R. Cosman, JR., Gloucester, MA (US)

(21) Appl. No.: 18/410,997

(22) Filed: Jan. 11, 2024

Related U.S. Application Data
(60) Provisional application No. 63/587,316, filed on Oct. 2, 2023.
(60) Provisional application No. 63/516,641, filed on Jul. 31, 2023.
(60) Provisional application No. 63/494,633, filed on Apr. 6, 2023.
(60) Provisional application No. 63/489,716, filed on Mar. 10, 2023.
(60) Provisional application No. 63/479,880, filed on Jan. 13, 2023.

(51) Int. Cl. A61B 18/12; A61B 18/00

(52) U.S. Cl. CPC A61B 18/1206; A61B 2018/00577; A61B 2018/00642; A61B 2018/00678; A61B 2018/00714; A61B 2018/00791; A61B 2018/00875

(57) ABSTRACT

This invention relates to systems and methods for tissue ablation using a moving-shot technique, including ablation of thyroid nodules and tumors, of uterine fibroids, of benign and malignant tumors in the body for a medical purpose, including radiofrequency ablation, microwave ablation, cryoablation, irreversible electroporation, laser ablation, and other ablation technologies. The invention includes user-interface elements that solve unmet needs for such tissue ablation, such under interface elements including an audible signal indicative of measured impedance, a historical graph of ablation apparatus output readings (including output power and impedance), a footswitch for hands-free activation and deactivation of the generator output (and possibly other controls), automatic coordination of ablation generator and probe-cooling pump outputs (including using cooled probe temperature as a parameter), automatic computation and display of information about the entirety of a multi-ablation procedure (including total energy delivered, average power delivered, total time of energy delivery), storage of procedure data (including generator readings and user annotations). The invention further includes guidance of the ablation probe using nerve stimulation for such tissue ablation.

### CLAIM OF PRIORITY

This application claims priority to U.S. Provisional Patent Application No. 63/479,880, filed Jan. 13, 2023, U.S. Provisional Patent Application No. 63/489,716, filed Mar. 10, 2023, U.S. Provisional Patent Application No. 63/494,633, filed Apr. 6, 2023, U.S. Provisional Patent Application No. 63/516,641, filed Jul. 31, 2023, and U.S. Provisional Patent Application No. 63/587,316, filed Oct. 2, 2023, each of which is incorporated by reference in its entirety.

### FIELD OF THE INVENTION

The invention relates to an electrosurgical system.

### BACKGROUND

Moving shot tissue ablation involves the creation of multiple ablation zones by moving an ablation probe to multiple locations in bodily tissue, to create a larger total ablation zone that conforms to the shape of a target anatomic structure, for a medical purpose. Ablation systems include radiofrequency, microwave, laser, cryogenic, irreversible electroporation, and other types of ablation systems. Ablation destroys tissue locally by means of elevated temperature, lowered temperature, electrical effects, and other physical processes depending on the type of ablation system. Moving shot ablation for the destruction or size reduction of thyroid nodules, uterine fibroids, and other type of cancerous and non-cancerous tumors that reside in close proximity to sensitive anatomical structures whose destruction is medically undesirable. Moving shot ablation is typically guided using ultrasound imaging to provide real-time imaging of the probe location and ablation process relative to target and non-target anatomical structures.

### SUMMARY

In one aspect, a system for radiofrequency tissue ablation can include a radiofrequency generator, a radiofrequency electrode, and a coolant pump; wherein the generator delivers a radiofrequency ablation signal the electrode, the radiofrequency ablation signal being adapted to ablate a tissue via the electrode; the generator is adapted to measure a power and an impedance of a radiofrequency ablation signal; the generator displays a graph of the measured impedance over time and a graph of the measured power over time; the generator produces an audible tone that indicates the value of the measured impedance; activation of the pump cools the electrode; deactivation of the pump stops cooling of the electrode; the system includes a cooled-RF mode of operation in which the generator delivers a radiofrequency ablation signal to the electrode and the pump cools the electrode.

In another aspect, a tissue ablation system can measure an impedance and sounds an audible signal, wherein, if the measured impedance is below an impedance-audio threshold, the generator configures the audible signal to be in a first form wherein a characteristic of the audible signal changes with changes in the measured impedance; and wherein, if the measured impedance is above the threshold, the generator configures the audible signal to be in a second form that is audibly distinct from the first form.

In another aspect, a system for ablation of tissue in the thyroid gland via an ablation probe can be adapted to deliver a nerve-stimulation signal to the ablation probe.

In certain embodiments, the electrode can further include a temperature sensor, and wherein the generator measures the electrode temperature.

In certain embodiments, the system can include a non-cooled mode of operation in which the generator delivers a radiofrequency ablation signal to the electrode, the pump does not cool the electrode, and the generator controls the electrode temperature.

In certain embodiments, in the cooled-RF mode of operation, the generator can deliver the radiofrequency ablation signal only if the electrode temperature is below a cooling temperature threshold.

In certain embodiments, the generator can display a graph of the measured electrode temperature.

In certain embodiments, the generator can be adapted to activate and deactivate the pump.

In certain embodiments, in the cooled-RF mode of operation, the generator can regulate the measured power of the radiofrequency ablation signal.

In certain embodiments, if the measured impedance is below an impedance-audio threshold, the generator can configure the audible signal to be in a first form wherein a characteristic of the audible signal changes with changes in the measured impedance; and wherein, if the measured impedance is above the impedance-audio threshold, the generator configures the audible signal to be in a second form that is audibly distinct from the first form.

In certain embodiments, the system can include a nerve-stimulation mode of operation in which the generator delivers a nerve-stimulation signal to the electrode.

In certain embodiments, the generator can include a cut-off control wherein, in modes of operations in which the generator delivers a radiofrequency ablation signal to the electrode, the generator automatically stops delivery of the radiofrequency ablation signal if the measured impedance rises above a relative impedance threshold, wherein the relative impedance threshold is adapted to be higher than a prior measured impedance value.

In certain embodiments, the prior measured impedance value can be the measured impedance at the time when delivery of the radiofrequency ablation signal was last initiated. In certain embodiments, the audible signal can be adapted to take at least three forms, wherein each form indicates that the measured impedance is in a different range of impedance values.

In certain embodiments, the graphs of impedance and power can be plotted on the same time axis.

In certain embodiments, the generator can be adapted to measure an energy of the radiofrequency ablation signal.

In certain embodiments, the generator can be adapted to measure a total energy of the radiofrequency ablation signal over more than one instance of delivery of the radiofrequency ablation signal.

In certain embodiments, in the generator can be adapted to distinguish measurements collected during heating periods from measurements collected during non-heating periods, heating periods being periods of time in which measurements indicate that delivery of the radiofrequency ablation signal to the electrode is materially increasing the size of an ablation zone in a tissue, non-heating periods being period of time in which the measurements indicate that delivering of the radiofrequency ablation signal to the electrode is not materially increasing the size of the ablation zone in the tissue; wherein the generator is adapted to display or record statistics about the value and timing of measurements in the heating periods; and wherein the generator is adapted to display or record statistics about the value and timing of the measurements in the non-heating periods.

In certain embodiments, the system can adapted for moving-shot radiofrequency tissue ablation in an organ selected from the list: thyroid, uterus, kidney, liver, lung, and an organ in the human body.

In certain embodiments, further including a footswitch, wherein the footswitch can be adapted to activate and deactivate delivery of the radiofrequency ablation signal to the electrode. In certain embodiments, the footswitch can be adapted to adjust a target parameter for delivery of radiofrequency ablation selected from the list: power, temperature, voltage, current, a parameter of the radiofrequency ablation signal.

In certain embodiments, the generator can include a data storage system that stores the measurements.

In certain embodiments, the footswitch can include a button, depression of the button activates delivery of the radiofrequency signal, and release of the button deactivates delivery of the radiofrequency ablation signal.

In certain embodiments, the system can further include an ultrasound apparatus that measures ultrasound data, and a display; wherein generator measurements and ultrasound measurements are displayed on the display.

In certain embodiments, the graphs of impedance, power, and electrode temperature can be plotted on the same time axis.

In certain embodiments, the generator can further include numeric displays of the measured power and the measured impedance, the numeric display and graph of measured impedance are drawn in a first color, and the numeric display and graph of measured power are drawn in a second color, the first and second colors are different.

In certain embodiments, the data storage system can store measurement from more than one ablation procedure.

In certain embodiments, the system can include a stimulation-guided mode of operation in which the generator delivers a nerve-stimulation signal and a radiofrequency ablation signal to the electrode at the same time.

In certain embodiments, the generator can be configured to limit the power of the radiofrequency ablation signal in the cooled-RF mode of operation in order to limit the likelihood that the measured impedance rises to exceed an impedance cut-off threshold.

In certain embodiments, the audible tone can also be an output indicator per IEC 60601-2-2.

Other aspects, embodiments, and features will be apparent from the following description, the drawings, and the claims.

### BRIEF DESCRIPTION OF DRAWINGS

FIG. 1A is a schematic illustration of an ablation system during moving-shot ablation in the thyroid gland.

FIG. 1B is a schematic illustration of an ablation system during ablation in the thyroid gland wherein the ablation probe is cooled.

FIG. 1C is a schematic illustration of an ablation system during ablation in the thyroid gland wherein the ablation probe is not cooled.

FIG. 1D is a schematic illustration of an ablation system during ablation in the thyroid gland.

FIG. 2A is schematic illustration of nerve-stimulation signal.

FIG. 2B is schematic illustration of combined nerve-stimulation and radiofrequency signal.

FIG. 2C is schematic illustration of circuit for generating nerve-stimulation a radiofrequency signals.

FIG. 2D is schematic illustration of circuit for generating nerve-stimulation a radiofrequency signals.

FIG. 3A is a method a nerve-stimulation-guided ablation in the thyroid gland.

FIG. 3B is a method a nerve-stimulation-guided ablation in the thyroid gland.

FIG. 4 is a method for cooled and non-cooled ablation in the thyroid gland.

FIG. 5 is a schematic illustration of a variety of RF ablation electrodes.

FIGS. 6A, 6B, 6C, 6D, and 6E are a schematic illustration of a moving-shot ablation process.

FIG. 7A is a schematic illustration of an audible signal that depends on impedance.

FIG. 7B is a method for moving-shot ablation that is guided by an audible signal.

FIG. 8 is method for stopping tissue ablation by means of a relative impedance threshold.

FIG. 9A is a schematic illustration of segmentation of a moving-shot ablation process into heating and boiling periods.

FIG. 9B is a method for characterization of heating and non-heating periods of a moving-shot ablation process.

### DETAILED DESCRIPTION

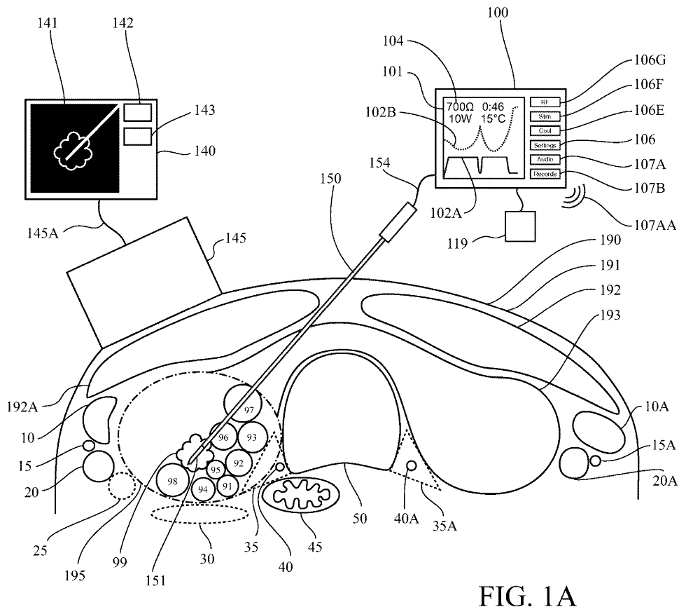

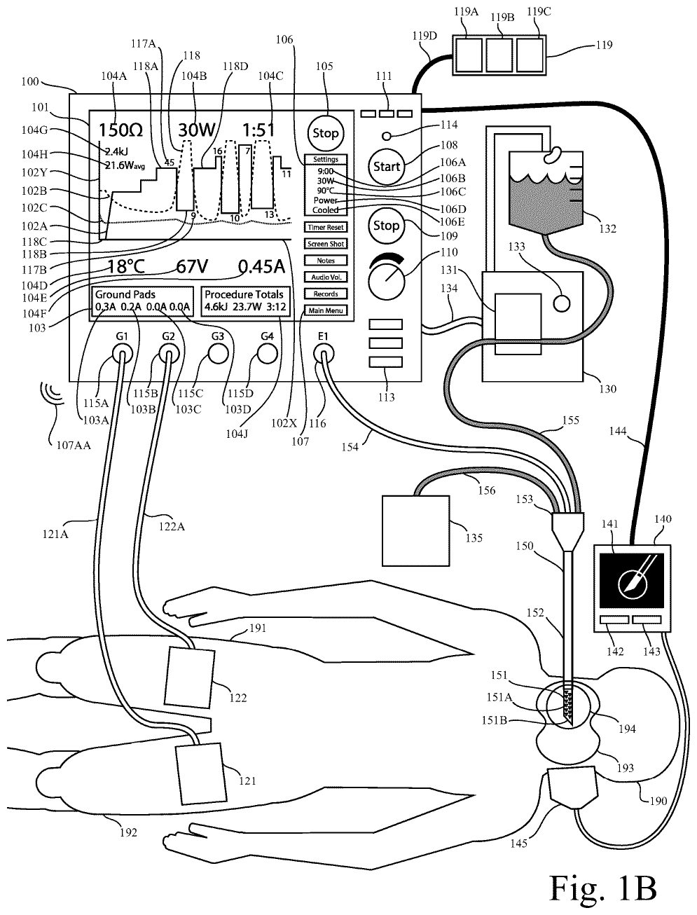

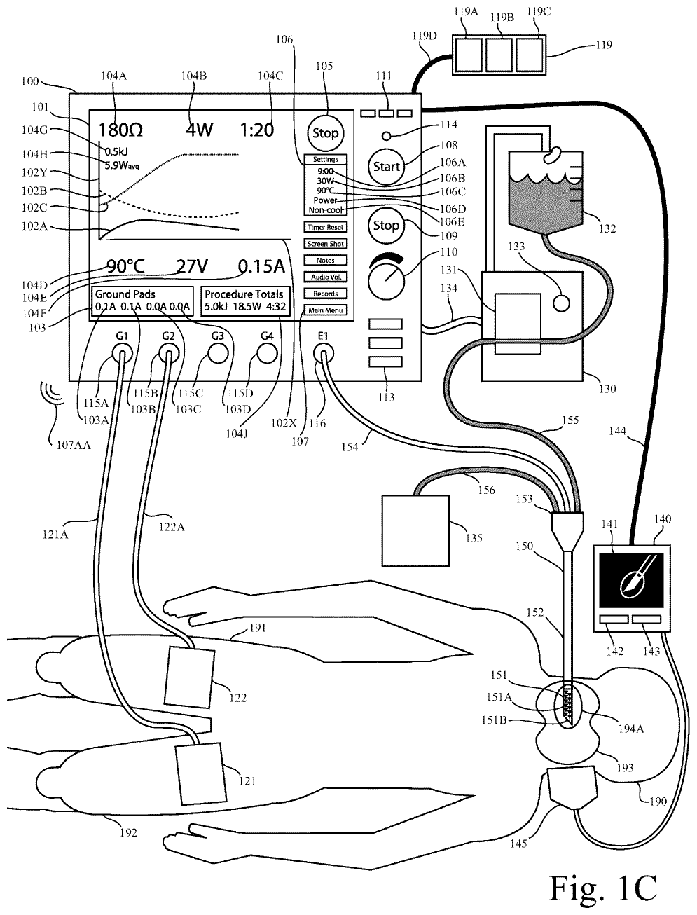

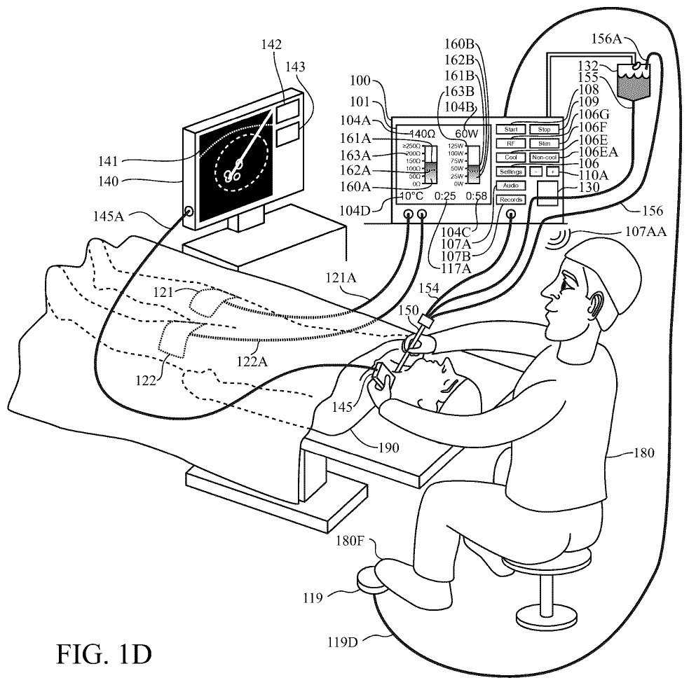

Referring to FIG. 1A, radiofrequency (RF) ablation (RFA) within the thyroid gland 193 (“thyroid ablation”, “thyroid RFA”) is used to reduce the size of benign thyroid nodules 195, to destroy malignant tumors 195, and other medical applications. In FIG. 1A, a transverse cross section of the thyroid gland 193 is shown in the frontal portion of the neck of a human patient 190, and thyroid nodule/tumor 195 is shown with a dash-dot outline within the thyroid gland 193, and an ablation probe 150 is inserted into the thyroid 193 and thyroid nodule 195 percutaneously, i.e. through the skin 191. In some cases, ablation is performed with the electrode in a single position, typically for small nodules. In some cases, ablation is performed by moving the electrode 150 to multiple positions and creating multiple ablations 91, 92, 93, 94, 95, 96, 97, 98, 99 (each of which if depicted as a circle in which the reference number is positions, except for ablation zone 99 that is depicted as a cloud-like region), either to target multiple separate target structures (e.g. nodules, tumors), or to create a single, ideally confluent, ablation zone that conforms to the size and shape of a single large target structure 195 (e.g. nodule, tumor), or both, as well as avoiding non-target structures (e.g. skin 191, muscle such as sternocleidomastoid carotid artery 10, medial-type vagus nerve 15, internal jugular vein 20, middle cervical sympathetic ganglion and/or posterior-type vagus nerve 25, recurrent laryngeal nerve 40, esophagus 45, trachea 50) of which ablation would cause undesired adverse effects (e.g. loss of function, voice change, bleeding, open wound, pain). The physician positions and moves the RF electrode 150 for such ablations, and controls the process of such ablations, using the impedance of the RF output signal measured by the RF generator 100, as well as real-time using ultrasound imaging 140. The impedance and ultrasound imaging 140 provide complementary information about tissue heating. Such multi-position RFA, particularly when use to conformally ablate a target structure using multiple adjacent or overlapping ablation, is commonly referred to as “moving shot” RFA, or RF ablation using a “moving shot” technique. In some cases, the “moving shot” technique involves moving the RF electrode to an adjacent nearby location (typically by withdrawing the RF electrode 150 by a length that is approximately equal the length of the metallic “active tip” 151, which is a conductive structure at or near the distal end of the RF electrode, and from which RF output conducts to tissue in which the RF electrode is positioned) without stopping the RF output delivery in response to impedance changes (i.e. the impedance rising above some threshold indicative of substantial tissue boiling around the electrode tip) and/or indications of substantial tissue boiling around the electrode tip in the ultrasound image (i.e. a dense hyperechoic region around the electrode active tip). In some cases, a “moving shot” technique involves stopping the RF output in response to impedance changes (i.e. the impedance rising above some threshold) and/or indications of substantial tissue boiling around the electrode tip in the ultrasound image (i.e. a dense hyperechoic region around the electrode active tip, which is depicted as the cloud-like online on ultrasound image 141 in FIG. 1A, and which corresponds to ablation zone 99 in the tissue), moving the RF electrode to a nearby location (either by withdrawing the electrode by approximately its active tip length, or by redirecting the electrode along another path through the tissue), and then restarting the RF output. For example, in the example shown in FIG. 1A, the ablation zones 91-99 can be created in numeric order, starting with ablation 91, and then ablation 92, 93, 94, 95, 96, 97 in succession, and finally ablation 99 which is actively forming around the distal tip 151 of the ablation probe 150. In some examples, additional ablations are created such that the tumor 195 is substantially fully ablated, or sufficiently ablated to have a clinical effect, such as size reduction or cancer treatment. Such an impedance threshold is often determined by the physician as a degree of increase in impedance from its initial value, e.g. an increase of 50 ohms, or an absolute impedance value that indicates substantial tissue boiling around the tip of the electrode. In some cases, the physician only moves the RF electrode when the output is off; and, in other cases, the physician moves the RF electrode when the RF output is on for some of the procedure, and when the RF output is off for some of the procedure. Thyroid ablation is generally performed using an internally-fluid-cooled RF electrode 150, using a set power 9 i.e. a target power level for the RF ablation signal) and using RF-signal impedance for feedback about the ablation processes. As such, the physician commonly (1) looks at the ultrasound image 141 to monitor the electrode and heat-related imaging artifacts (e.g. hyperechoic image of high-temperature bubbles 99); (2) monitors the generator impedance reading 104 whose decrease is indicative of tissue heating and whose rapid, large increase is indicative of boiling around the RF electrode tip 151; (3) holds and moves the RF electrode 150 with one hand; (4) holds the ultrasound transducer with the other hand; (5) repeatedly starts and stops the RF generator output to reposition the electrode 150, and to restart the RF generator output after the generator output turns off due to automatic high-impedance cut-offs (“open circuit” output cut-offs); (6) adjust the RF generator output level based on the timing of impedance-rises with delivered energy (a impedance-rise-time in the range of 5-30 seconds are common, but can be longer, based on physician preference).

One clinical risk of thyroid ablation is damage to nerves (e.g. 15, 40) in or within the thyroid gland 193, such as the recurrent laryngeal nerve 40, superior laryngeal nerve, inferior laryngeal nerve, and branches thereof. Other such nervous structures include vagus nerve 15, the middle cervical sympathetic gangion, and/or vagus nerve 25. Such nerves may have variable position across patients; for example, region 30 (having a dashed outline) shows exemplary locations in which the recurrent laryngeal nerve may be located in different people. Such nerves and other sensitive structures can be located on both the right and the left side of the thyroid gland, for example, vagus nerve 15, 15A; recurrent laryngeal nerve 40, 40A; danger triangle 35, 35A; carotid arteries 10, 10A; internal jugular veins 20, 20A. Damage to such nerves can cause lasting harm, such as voice change, hoarseness, swallowing issues, sensory issues, and muscle denervation. One objective of the present invention is to avoid nerve damage during thyroid ablation.

Clinical risks of thyroid ablation include damage to sensitive tissue in or near the thyroid 193, such as nerves 15, 20, 40; skin 191, muscle 192; trachea 50, blood vessels 10, 20; esophagus 45. The “danger triangle” 35 (shown in FIG. 1A as a triangle having a dashed outline) is a region that may include the recurrent laryngeal nerve 40 and esophagus 45. Damage to such structures can cause lasting and serious damage, possibly requiring hospitalization or surgery, such damage including burns, infections, pain, trouble breathing, trouble swallowing. One objective of the present invention is to avoid damage to sensitive structures during thyroid ablation.

A challenge of thyroid RFA is the physician's need to monitor and respond to both the ultrasound image 140 and the generator 100 readings, which are typically displayed as real-time numeric readings on the generator display; both the ultrasound display and such numeric readings are constantly updated in real-time without capturing any history of their values on-screen (or even in an persistent procedure record), so the physician must be looking at this as they occur in order to see them during treatment. This is a complexity that increases uncertainty and can compromise the physician's responsiveness during treatment. It is common for physicians to have their assistant(s) call out impedance readings verbally so that the physician can watch the ultrasound machine 140. This is a burden on the assistants, who have other responsibilities during the medical procedure, and a financial and staffing burden on the physician. One objective of the present invention is to address this challenge.

A challenge of thyroid RFA, of moving shot ablation in general, is the physician's need to start and stop the generator output repeatedly, and intermittently adjust the generator output level, whilst having both hands occupied with the RF electrode 150 and ultrasound transducer 145. It can also be desirable to stop the fluid pump (in the example shown in FIG. 1A, the fluid pump 130 is included in generator 100, and its associated fluid tubing 155, 156 is included in generator-to-electrode cable 154) that is fluid-cooling the RF electrode 150 at the same time that the RF output of generator 100 starts and stops. This is a complexity that increases uncertainty and can compromise the physician's responsiveness during treatment. It is common for physicians to have their assistant(s) start and stop the RF generator 100, and possibly the fluid pump 130, when requested by the physician. This is a burden on the assistants, who have other responsibilities during the medical procedure, and a financial and staffing burden on the physician. One objective of the present invention is to address this challenge.

Radiofrequency ablation of uterine fibroids (including, without limitation, leiomyomas and myomas) can utilize similar equipment and techniques (e.g. ultrasound guidance and moving shot technique) and presents similar technical, medical, and other challenges (e.g. conformal ablation, avoidance of nearby sensitive structures) to thyroid RFA. Such uterine ablation can be performed transvaginally or transcutaneously. One objective of the present invention is to address the challenges of uterine fibroid ablation, ablation of other bodily structures that use ultrasound guidance and the moving shot technique, and ablation of other bodily structures. It is understood that the embodiments set forth in this invention (including, without limitation, systems and methods) and described in relation to thyroid ablation, can also be applied to uterine fibroid ablation, and to ablation of other bodily structures (such as liver, kidney, lung, bone, soft tissue, pancreas, spleen) for a medical purpose.

In addition to RFA, embodiments of the present invention can be applied to other tissue ablation technologies, including, without limitation, microwave ablation (MW), irreversible electroporation (IRE), laser, and cryo ablation. For example, in some examples, the generator 100 can be a MW generator and pump, and the ablation probe 150 can be a MW antenna.

As shown in the embodiments shown in FIG. 1A, generator 100 includes a display 101, controls 106 106E 106F 106G 107A 107B, a footswitch 119, and implicitly a fluid pump 130 with fluid source 132 and sink 135 for cooling ablation probe 150. The display 101 includes numeric readings 104, which in FIG. 1A, include impedance, power, time, and temperature; whose respective values at the exemplary moment shown in FIG. 1A are 700Ω, 10 W, 0:46, and 15° C.; and whose respective units of measure are ohms, watts, minutes:seconds, and degrees centigrade. Display 101 includes graphical display of certain readings over time, which in FIG. 1A are power output 102A and impedance 102B. In the example of FIG. 1A, the graphs 102A and 102B show the time course of the readings during moving-shot ablation at two consecutive positions of the electrode 150 that produced ablation regions 98 and 99, during which the operator did not turn off the RF ablation output when pulling the electrode tip 151 back from position 98 to position 99, and during which the target power was constant (e.g. a value in the range 5-120 Watts) but decreased (e.g. to a value in the range approximately 1-10 Watts) after sustained heating at each position due to gas formative around the probe tip 151 and a consequent large rise in impedance 102B and limited RF voltage output of the generator 100 (in accordance with Power=Voltage*Current and Current=Voltage/Impedance, Power=Voltage*Voltage/Impedance is an approximate model for RF tissue heating). Generator 100 includes an audio speaker and user control 107A that produces an audible signal 106AA that is indicative of the impedance reading 104. The footswitch 119 can be used to turn the generator output on and off; for example, the electrode output turns on when the footswitch 119 is depressed, and the output turns off when the footswitch 119 is released. The generator includes a circuit and user-control 106G to activate or deactivate delivery of an RF-ablation output signal to the electrode 150. The generator includes a circuit and user-control 106F to activate or deactivate delivery a nerve-stimulation output signal to the electrode 150. The generator includes a pump 130 and user-control 106E for activation or deactivation of cooling of the electrode 150. The generator 100 includes user control 106 for various generator settings, controls, and functions, including, without limitation, the target power, the maximum output time, thresholds for impedance above and below which the generator output will turn off or not activate, target temperature, control mode, parameters and thresholds affecting the audio signal 106AA, thresholds for temperature above and below which the generator output will turn off or not activate, settings for electrode cooling, settings adjusting the readings and graphs, settings adjusting functions of the footswitch 119, settings and controls for remote control of the ultrasound machine 140 by means of the user interface of the generator 100, settings for storage of procedure data to internal memory, controls for storage of screenshots of the display 101. The generator includes internal memory, means of data export, and user-control 107B for storage of procedure data, including, without limitation, settings, readings, and user annotations. Such user annotations can include information about the patient, doctor, hospital or clinic, time and date, target organ, ablation target type and characteristics, and other clinical or technical information about an ablation procedure, including those that can be entered using a keyboard or numeric keypad. The generator 100 is adapted to save automatically all procedure data for multiple ablation procedures (e.g. the most recent 1-1000 or more ablation procedures) for latter recall and analysis. Such automatic and a persistent storage of procedure can by generator 100 can greatly facilitate medical record keeping, later analysis of technique, later analysis and organization for medical publication, particularly in a busy medical practice where many ablation procedures are frequency performed, and particularly for moving-shot ablation procedures during which the attention of the physician and staff is occupied with many medical functions such that record keeping is challenging.

Referring now to FIG. 1B¬, another embodiment of the present invention is shown that includes an RF generator 100; one or more ground pads 121, 122 applied to the skin surface of patient 190; an ablation probe 150 placed within target anatomy, such as the thyroid gland 193 or a tumor or nodule therein; a coolant pump 130 that is configured to cool one or more of the ablation probe 150 by drawing coolant (such as saline or water) from source 132; a reservoir 135 for collection of used coolant; and an ultrasound machine 140. The ground pads 121, 122 are connected at jacks 115A, 115B, respectively, to a reference potential generated by the generator 100 to carry return currents from the ablation probe 150, which is connected to the electrode-output potential (which can be an RF ablation potential and/or a nerve stimulation potential) delivered via connector 116. In some examples, the ground pads 121,122 can be omitted when the probe 150 is a bipolar electrode (i.e. the electrode includes a contact connect to the reference potential and a contact connected to the electrode-output potential). The at least one ablation probe 150 can be connected to the electrode output jack 116 of the RF generator 100. The coolant pump 130 can be operably connected to the generator 100 by means of control connection 134, and the activation, deactivation, and rate of coolant flow to an ablation probe can be controlled by the generator 100, for example, in coordination with ablation programs being run by the generator 100 or with operational modes of generator 100. The ultrasound imaging machine 140 includes user controls 142 and can be operably connected to the generator 100 by means of control connection 144. Controls 113 included in the generator 100 can be used to operate some or all functions of the ultrasound machine 140. Controls 143 included in the ultrasound machine 140 can be used to operate some of all of the function of the generator 100. A data file that includes procedure data both from the RF generator 100 and the ultrasound machine 140 can be produced by the combination of the generator 100 and the ultrasound machine 140; that data file can be saved to internally memory included in the generator 100; that data file can be saved to internal memory included in the ultrasound machine 140; and that data file can be exported to an external data repository, such as an external disk or computer, by means of a data connection included in the either the generator 100, the ultrasound machine 140, or both. In some other embodiments, the ultrasound machine 140 and generator 100 can be integrated into a single housing. In some other embodiments, the ultrasound machine 140 and generator 100 can be integrated into a single chassis with a single screen that includes information displayed both on generator display 101 and on ultrasound display 141.

The generator 100 includes a user interface, including a graphical display 101 which can be a touch screen, and includes numerical displays 104A, 104B, 104C, 104D, 104E, 104F, 104G, 104H, 104J and graphical displays 102A, 102B, 102C, of readings and controls. The graphical user interface 101 can graph readings, such as elapsed time, impedance, current, temperature, voltage, and power, as a function of time. In the embodiments shown in FIG. 1B, the graphical displays 102A, 102B, 102C each plot a parameter on the vertical “y” axis 102Y, as a function of time on the horizontal “x” axis 102X, and can each plot be referred to as an (x,y) line plot where x is time and y is a parameter selected from the list: impedance, current, power, voltage, temperature, other generator reading, other measurement by the generator 100. Each graphic display 102A, 102B, 102C and numerical display 104A, 104B, 104C, 104D, 104E, 104F, 104G, 104H, 104J of a parameter can be color-coded so they are easy to discriminate from each other on the computer graphic display 101. Each digital and graphic display of the same reading can have the same unique color, so that it is easy for the user to associate the digital and graphic display of the same parameter, and to distinguish displays of different parameters. For example, impedance reading 104A can have the same color as impedance graph 102B in FIG. 1B, and that color can be green. The power reading 104B can have the same color as power graph 102A in FIG. 1B, and that color can be orange. The temperature reading 104D can have the same color as temperature graph 102C in FIG. 1B, and that color can be red. In other embodiments, other color assignments can be used. The generator 100 includes one or more controllers for automated and/or semi-automated control of the RF output, including for example, control of the RF output signal as a function of its measured power, current, voltage, temperature, impedance, and/or the past variations of such readings.

The RF generator 100 can include a lamp 114 that indicates the active delivery of generator output to the electrode 150; a mechanical button 108 by means of which a user, such as a medical doctor or nurse under the direction of a doctor, can turn on the electrode output; a mechanical button 109 by means of which a user can turn off the electrode output; a mechanical knob 110 by means of which the user can adjust the output level of the electrode output or a settings that affects electrode output (such as the target RF power, target electrode temperature, or stimulation level); controls 113 for an ultrasound imaging machine 140; one or more data connections 111, such as USB ports, ethernet ports, or wireless connections, that can each provide for transfer or data and/or control connection with one or more of a computer network, an external hard disk, a computer, a flash drive, a wireless remote control, a wired hand controller, or printer; a jack 116 that provides delivery of generator output to one or more ablation probes 150; one or more ground pad jacks 115A, 115B, 115C, 115D for connection to one or more ground pads 121, 122; and a touch screen display 101. In some embodiments, the mechanical knob 110 can take other forms as an alternative or an additional to the mechanical knob 110, such forms including mechanical up and down buttons, on-screen up and down buttons, sliders, and other user interface elements for adjusting a value up and down.

The generator 100 can store all measured readings during, before, and after running an ablation program to memory as a procedure record file or files (which can be accessed, edited, or exported via the “Records” control in button group 107), which can include images of the display 101 that can be saved via the “Screen Shot” control in button group 107. The record file can be stored in memory internal to the generator and to external memory, such as an external disk attached to data port 111. The procedure record can include averages, maximum, modes, and other statistics of measured readings configured to provide the physician with a meaningful, easy to understand the record of a medical ablation procedure. The procedure record can include or be processed to produce graphs and analysis of measured values. The procedure record can include text annotation of the data, including patient information (age, sex, date of birth, name, identification number), information about the size and type of target structures (organ, pathology, tumor type, malignancy or non-malignancy, vascularity), information about the doctor (name, department, hospital/clinic name), other information, such text annotation being accessible via the “Notes” control in button group 107. In some embodiments, the generator can explicitly prevent the storage export of sensitive patient information in the procedure record. The procedure record can include ultrasound, fluoroscopy, CT, MRI, and other imaging data.

In some embodiments, an interface between the ablation apparatus 100 and ultrasound (US) machine 140 via connection 144 can be standardized to allow for interoperability different generators 100 and different ultrasound machines 140. The interface 144 can carry multiple type of a data, including data for control of operations of the generator 100, data for control of operations of the US imaging system 140, data representing operation and measurements of the ablation apparatus 100, data representing operation and measurements of the US imagining machine 140, such that generator displays can be shown on the ultrasound machine, and/or ultrasound displays can be shown on the generator screen 101, for user convenience and to facilitate moving-shot ablation. In some examples, the communication connection 144 can be bi-directional, wherein data is sent back and forth between the electrosurgical system 100 and the ultrasound imaging apparatus 140. In some embodiments, connection 144 can be wired. In some embodiments, connection 144 can be wireless. In some embodiments, the ultrasound imaging apparatus 140 can be another type of medical imaging apparatus, including, without limitation, a fluoroscopy imaging apparatus, an x-ray imaging machine, an MRI scanner, a CT scanner, a spiral CT scanner, a PET scanner, an optical coherence tomography (OCT) device. In some embodiments, the medical apparatus 100 can be another type of ablation interventional medical device, including, without limitation a RF generator, a MW generator, a laser ablation device, an irreversible electroporation (IRE) ablation apparatus, a cryo-ablation device.

The graphical plot of parameters 102A, 102B, 102C gives the clinician a visual, intuitive, and real time update and evaluation of whether the ablation process is proceeding properly and safely, as well as a persistent visual history of that process that allows the physician to review the process, even if the physician must turn his or her attention away from the generator display intermittently, for example, to attend to the patient or other information sources or control relevant to the procedure (such as those of the ultrasound apparatus 140) or to move the electrode 150, as is common in moving-shot ablation techniques. Without loss of generality, FIG. 1B shows, and this text describes, exemplary graphs, readings, and settings at one point during a thyroid ablation. In the example shown in FIG. 1B, graph 102A (drawn in a solid line) plots the RF output power, graph 102B (drawn is a dashed line) plots the impedance between the two RF potentials generated by the RF generator 100 (i.e. the potential at the electrode 150 and electrode connector 116, and the reference potential at the ground pads 121, 122 and the ground pad connectors 115A, 115B, 115C, 115D), and graph 102C (drawn in a dotted line) plots the temperature measured at the electrode tip 151 on vertical axis 102Y, all relative to the time axis 102X. In other examples, graph 102A can plot another measure generator output level, such as voltage or current. These graphs over time show an exemplary history of the output power, impedance, and temperature during one instance of RF output delivery, which is ongoing, during a thyroid ablation procedure. The graphs for power 102A, impedance 102B, and temperature 102C, and the numerical readings for power 104B, impedance 104A, and temperature 104D are updated substantially in real-time (e.g. several times per second) such that the most recent (rightmost) point on each graph corresponds to the present numerical reading. The initial readings when the RF output is first turned on by the user are plotted at the left of the time axis. When the output first turns on, the generator holds the RF power level very low 118 until the temperature 102C drops (from body temperature or a higher temperature due to prior tissue heating) to a level that indicates sufficient RF electrode cooling, for example, a value of at most 30° C., or preferably a value in the range 0-20° C. for fluid-cooled RF ablation. Once the temperature indicates sufficient electrode cooling, the output level ramps up to the initial target power set by the user, e.g. 20 watts. In response to the applied power, the impedance 102B drops due to increased ion mobility in the tissue. If tissue around the electrode is not reaching a boiling point fast enough, as ascertained by bubble formation on ultrasound imaging 141 and/or a rapid and marked increase in impedance 10B and/or a drop in RF output power in spite of high output RF voltage, the user increases the target power 106B by means of power control 110, as depicted in FIG. 1B by the two upward steps (e.g. in 5 W increments, from 20 W to 25 W, and then from 25 W to 30 W), in the power plot 102A during the first high-power pulse 118A. This energy delivery causes significant boiling around the electrode tip and a rapid rise in impedance 102A, as shown by peak 118 in FIG. 1B. The boiling is sustained due to continued energy delivery such that the impedance 102B reaches a high level (e.g. 250-2000 ohms or more, or possibly less, depending on electrode geometry and tissue characteristics), but in this example, the impedance level of peak 118 does not exceed the maximum-impedance cut-off threshold set on the generator 100. In this example, the impedance graph value 102B is vertically saturated (e.g. at a value in the range 150-250 ohms, or at a value modestly higher than the typical non-boiling-tissue impedance range) so that variations in impedance at lower impedances typical of non-boiling tissue (e.g. typically less than 250 ohms) can be viewed by the user and used to control the ablation process; such focusing of impedance-graph resolution on the non-boiling range has an advantage for tissue ablation, and in particular moving-shot ablation, because the impedance during the non-boiling phases of tissue heating are used by the physician to control the electrode position. As the impedance spikes upward 118, the power output drops (as shown by low-point 118B on the power plot 102A) because the generator output reaches a maximum voltage; this power drop with sustained output voltage can be an indication of tissue boiling; the maximum voltage of the generator can be limited (either uniformly or for higher impedances) such that tissue heating will continue without tending to drive the tissue impedance above the generator's open-circuit cut-of threshold (i.e. maximum impedance cut off threshold) to facilitate moving-shot ablation wherein the physician desires that the RF output does not shut off between ablation positions. The generator detects the drop in power (e.g. from 118A to 118B) and/or the rise in impedance (118) to mark an end to a first “heating period” of the instance of RF output delivery, and the generator 100 displays the duration of this first heating period as reading 117A (which in this example, reads “45” seconds), which is displayed, in this example, just above the first heating period 118A. The duration of the first heating period 117A is the time elapsed since the later of (a) the last time the RF output was activated by the user, and (b) the end of the last non-heating period, as determined by a drop in impedance from a non-heating/boiling range to a heating/non-boiling range, achievement of a power output level indicative of substantial tissue heating, or another indicator of the end of a period in which the generator output is not likely to substantially heat tissue and/or expand the size of the ablation zone. The end of the first “heating period” 118A, starts the first “non-heating period” 118B during which the impedance is in a high range indicative of complete (or impeding complete) boiling around the electrode tip 151 and/or a drop in power below a level capable of sufficient expanding the ablation zone size (e.g. less than 15 watts for an RF electrode whose tip 151 is 18 gauge and 1 cm in length, or a value that depends on the electrode tip size). The generator 100 discriminates each “heating period” (i.e. a period of relatively low impedance indicative of non-boiling tissue wherein the generator can deliver RF power sufficient to substantially heat the tissue and expand the ablation zone size) and each “non-heating period” (i.e. a period wherein the generator 100 does not deliver RF power sufficient to substantially heating tissue, e.g. a “boiling period” in which boiling gas bubbles densely surround the electrode tip 151 such that impedance is relatively high and indicative of such boiling) during the present instance of RF output delivery, and the generator 100 indicates each such heating and non-heating periods' durations on the display 101, as depicted in FIG. 1B as exemplary durations “45”, “9”, “16”, “10”, “7”, “13”, “11” seconds positioned adjacent to the end of each such heating and non-heating period. During the non-heating period, when the user withdraws the electrode 150 to heat an adjacent portion of tissue without turning off the RF ablation output, impedance 102B again drops to a non-boiling level and the output power 102A returns to its target value (as shown by graph portion 118D), thus ending the first “non-heating period” 118B, whose duration is 9 seconds in FIG. 1B, and starting the next “heating period” 118D, whose ultimate duration is 16 seconds in FIG. 1B. This process continues through two more “non-heating periods” and two more “heating periods” shown in the on-screen graphs of FIG. 1B. In the power graph 102A, examples of the user's increasing and decreasing the power output level 106B, both during “heating periods” and “non-heating periods” in response to the duration of “heating periods” and ultrasound imaging 141. The electrode temperature graph indicates a cool temperature throughout the depicted instance of RF output delivery, approximately 18 deg C. as indicated by temperature reading 104D, because the electrode 150 and its indwelling temperature is being cooled by fluid circulated through its shaft by pump 130. An important advantage of the graphs 102A, 102B, 102C and of heating-period statistics 117A and non-heating-period statistics 117B is that they provide persistent displays of information about the ablation process to the user, even if the user's attention is focused on the patient or ultrasound machine 140 at times during that ablation process. This is a particular advantage during moving shot ablation (e.g. in the thyroid and uterus), and other ablation methods in which the ablation probe is moved during ablation energy delivery, because the physician must look at real-time imaging 141 while moving the electrode 150, and monitor the patient 190 as well. In addition, the graphs 102A-C, heating period durations (e.g. 117A), and non-heating period durations (e.g. 117B), remove the need for the user memorize past values and do mental calculations to determine changes, rates of change, and differences between numeric readings. If the generator 100 only included numeric readings 104A, 104B, 104C, 104D, 104E, 104F that change substantially in real-time, their values at certain time may be missed when the user is not looking at the generator 100; determinations of differences and rates of change may be difficult, inaccurate, or impossible to assess; and thus, user response to those values may be delayed. Such uncertainties and lead to more difficult, slower, less accurate, more conservative ablation treatments, ultimately resulting in over treatment, under treatment, increased surgical risks (such as risks from infection, pain, and additional anesthetics).

Audio tone 107AA can indicate to the physician operator information about variations in the impedance signal 104A as depicted by graph 102B without the physician's having to look at generator display 101. An audio tone 107AA that varies with impedance 104A, such as a tone whose carrier frequency is a monotonically increasing function of the impedance reading 104, can allow the physician user to hear rises and falls in the impedance signal, such as those depicted by 102B. The physician can hear heating of the tissue as the audio signal's 107AA tone-frequency drops with a drop in the impedance value 104A. The physician can hear prolonged or increased heating of the tissue as the audio signal's tone-frequency later rises with a rise in the impedance value 104A. The physician can hear tissue boiling as the audio signal's 107AA tone-frequency tone-frequency rapidly and greatly increases with a rapid and great increase in the impedance value 104A. An audio tone 107AA that additionally includes a distinct feature indicating that the impedance has exceeded a threshold that indicates tissue boiling can provide additional unambiguous information to the physician user in relation to tissue boiling and the completion of RF ablation process at a location. Such a feature can be a special tone, a tone in a special frequency outside the normal range of impedance-based tones, a pulsed tone, a double beep, or other type of audible waveform that is audibly distinct from the waveforms included in signal 107AA to indicate impedance value. For example, audio signal 107AA can indicate peaks in impedance, such as 118, that would prompt the physician user to move the ablation probe 150 in a moving-shot technique. In other embodiments, audio signal 107AA can be adapted to provide information about other generator readings, including impedance, power, time, temperature, voltage, and current. For example, audio tone 107AA can provide information about the elapsed duration of ablation output or of the most recent heating period, for example, including a distinct tone at regular intervals such as 1, 5, 10, 15, 20, 25, 30, or more seconds, or an interval that is clinically relevant or relevant to the physician user's desires. For example, if the physician user desires that tissue around the electrode boils within 30 seconds, the time-based tone 107AA can be set to 30 second intervals so the physician knows that this duration has passed without having to look at the generator timer 104C.

Shown in FIG. 1B are digital displays of the temperature 104D, current 104F, power 104B, voltage 104E, impedance 104A, the elapsed time since electrode output has been delivered since it was last initiated 104C, the energy delivered to the electrode since the electrode output was last initiated 104G, and average power delivered to the electrode since the electrode output was last initiated 104H. Display area 104J, labeled “Procedure Totals” includes digital displays that apply to all electrode output since the current medical procedure was started, not just since the electrode output was last started (as is displayed by readings 104A through 104H, and by graphs 102A, 102B, and 102C). The electrode output can be started and stopped multiple times during a single medical procedure, particularly for “moving shot” ablation procedures, and computation of such total-procedure values 104J can be useful to characterize the entire procedure and can be difficult to compute or estimate manually and accurately due to reading variations and the sheer volume of data during an ablation procedure. In FIG. 1B, area 104J includes numeric displays of the total ablation energy delivered to the electrode output, the average power delivered when the electrode output is active, and the total time for which the electrode output has been active; the respective exemplary values shown at the instant depicted in FIG. 1B are 4.6 kJ (kilojoules), 23.7 W (watts), and 3:12 (minutes:seconds). In one example, as shown in FIG. 1B, the current to each of the surface ground pads are shown digitally 103A, 103B, 103C, and 103D. In another embodiment, a plot of each of the ground pad currents over time is additionally shown on display 101.

FIG. 1B also shows graphic user interface controls, including button groups 105, 106, and 107. In one example, these controls include interaction with the automatic controller, computer graphic control, and data system to adjust parameters of the ablation process to suit the clinician needs. Button 105 allows the user to start and stop the electrode output, displayed “Stop” while the output is active, and displayed “Start” when the output is inactive. Control 106 includes a button to adjust generator settings and a display of selected output settings, including those for maximum output time 106A, set power 106B, set temperature 106C, control mode 106D (i.e. the principle parameter being controlled, e.g. power, voltage, current, temperature, impedance), and electrode cooling (or lack thereof) 106E, whose respective exemplary values shown in FIG. 1B are 9:00, 30 W, 90° C., Power (i.e. regulate the power of the RF ablation signal delivered to the electrode), and Cooled (i.e. via connection 134, the pump 130 is activated when the ablation output of generator 100 is activated in order that the pump 130 cools the electrode when RF ablation signal is delivered to the electrode, in contrast to an alternate setting value “Non-Cooled” for which the pump 130 is inactive and does not cool the electrode when RF ablation signal is delivered to the electrode, the setting value being adjustable by a setting for electrode cooling 106E).

The coincidence of a set-power setting and a set-temperature setting can be useful for cooled-RF ablation systems to allow the user to easily switch between power-controlled cooled-electrode ablation (which tends to create ablation zones that are larger and grow more quickly) and temperature-controlled non-cooled ablation (which tends to create ablation zones that are smaller and grow more slowly) either (1) for needle track coagulation after cooled-electrode ablation of a malignant tumor to avoid seeding of the needle track with tumor cells due to withdrawal of the ablation probe 150 from the tumor, or (2) to provide easy variation of ablation size, consistency, and control when ablating around sensitive structures (particularly when using a moving shot method), as is characteristic of ablation in the thyroid gland (see FIG. 1A) and other parts of the body. Cooled-electrode ablation zones tend to be larger, faster, and have less consistent size and less regular border because more power can be delivered to the tissue due the electrode cooling, and because boiling gas bubbles are created in the tissue that can develop and move irregularly. Non-cooled-electrode ablation zone tend to be smaller, slower, and have more consistent size and more regular borders because less power is delivered to the tissue because the maximal tissue temperature can be measured at the non-cooled electrode and controlled below boiling, and because longer application times allow more thermally conductive smoothing of peri-electrode temperatures. Generator 100 can provide easy selection between cooled and non-cooled ablation when the RF ablation output is initiated by the user, for example by means of a dialog that provides buttons for such selection, by means of button(s) on the main procedure display 101 (such as 106E in FIG. 1A, or buttons 106E and 106EA in FIG. 1D), or other user interface elements.

In some examples, settings control 106 allows the user to activate and deactivate nerve-simulation signal output to the electrode 150 from generator 100. In some examples, settings control 106 allows the user to adjust cut-off controls based on measured impedance, whereby the generated output is turned off, or does not turn on, if the impedance is above or below a threshold value; such a control can be a specified impedance value, an impedance value relative to a past impedance measurement, or a change in impedance value. Button group 107, in the example shown in FIG. 1B, includes a control labeled “Timer Reset” for resetting output timer 104C to zero, a control labeled “Screen Shot” for storing an image of display 101 to the current electronic procedure record, a control labeled “Notes” for annotating the current electrode procedure record, a control labeled “Audio Vol.” for adjusting the volume of audible signals (such as 107AA0 generated by the generator 100, and a control labeled “Main Menu” for stopping the current procedure, completing its associate electronic record file, and return the generator display 101 to a menu with general and administrative system functions and settings.

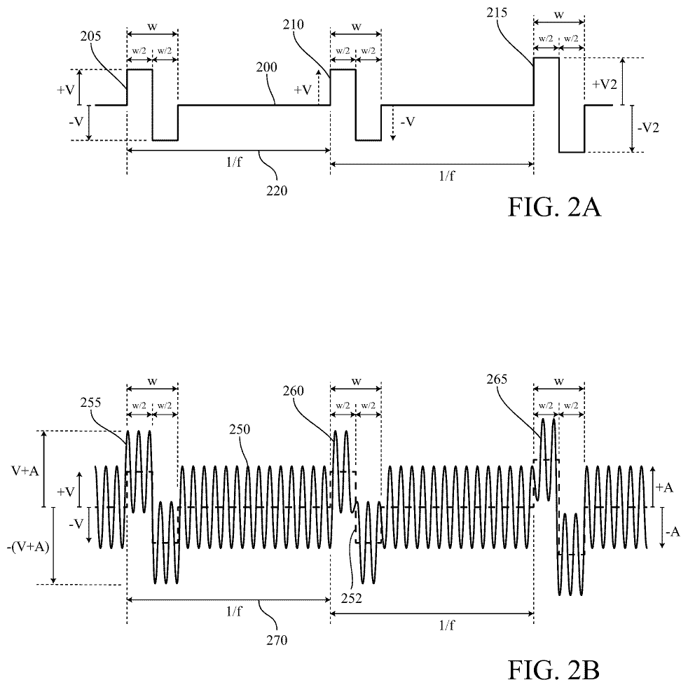

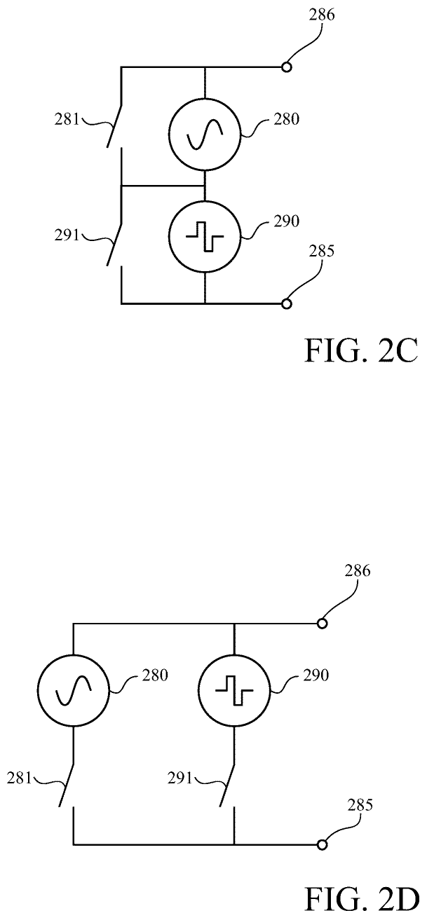

FIG. 1B also shows an example of a footswitch 119 connected to generator 100. In this example, footswitch 119 includes three buttons 119A, 119B, and 119C. Button 119A allows the user to turn on and turn off the electrode output (which can be an ablation output, a nerve-stimulation output, or both depending on generator settings or mode, adjusted by control 106 and other controls). Button 119A preferably activates the output when the button 119A is depressed, and deactivates the output when the button 119A is released, because this operation is intuitive and it avoids unintended and unsafe extension of output activation (and potential unintended tissue damage) due to the physician's losing track of the location of the footswitch after activating the output and being unable to actuate the button 119A timely when the physician desires to terminate the electrode output; this is particularly useful for moving-shot ablation methods which can involve many, frequent instances of turning the generator output on and off in a single medical procedure. In other embodiments, the electrode output starts when 119A is depressed and released when the output is not already running; and the electrode output stops when 119A is depressed and released when the output is already running. In other embodiments, other user interfaces for 119A are possible. Button 119B allows the physician user to increase the electrode output level, such as the RF output power or the nerve-stimulation signal amplitude (e.g. V in FIG. 2), and Button 119C allows the physician user to decrease the electrode output level. In one exemplary embodiment, each press and release of button 119B increases the output level by an increment, and each press and release of button 119B decreases the output level by a decrement. For example, such increment and decrement can be 5 watts or another value appropriate to the type of output level being adjusted. A footswitch 119 typically sits on the floor and its buttons are actuated by means of the user's foot.

Referring to FIG. 1B, FIG. 1B is a schematic drawing showing one example of an arrangement of an apparatus for performing high-frequency (HF) ablation (i.e. ablation by means of a high-frequency electrical signal, such as RF, MW, or other electrosurgical signal) of bodily tissue of the patient 190, in accordance with some¬¬proximal end, and an elongated shaft portion with a distal end and a proximal end, wherein the distal end is inserted into the patient body 190 percutaneously, and wherein the shaft portion includes an insulated portion 152 and a tip portion 151 at the distal end. In one example, the shaft portion can comprise rigid metal tubing which is insulated on its outside surface on the insulated portion 152 and uninsulated on active tip portion 151. In one example, the probe tip point of the probe tip is a trocar point (e.g. point 501A of electrode 500), and in other examples, the probe tip point can take other shapes known in the art of medical needle tip points. In other examples, the probe 150 can be a two-piece electrode-cannula system, wherein a cannula (e.g. 530) is inserted into the tissue and a separate electrode (e.g. 560 or 520) is inserted into the cannula 530 to form a combined electrode 150; these example have advantage for thyroid ablation, and for moving shot ablation, because the cannula provides a pathway for infusion of fluids (such as anesthetic for pain control during ablation, steroids for inflammation and pain control, DW5—5% dextrose solution—for thermal isolation of non-target structures from ablation zone) without requiring additional penetration of the skin with a separate needle. In another example, the shaft portion of the electrode 150 is a flexible structure having uninsulated tip portion 151; this has the advantage of uterine ablation, and other moving shot ablation, because it facilitates transvaginal insertion of the ablation probe into the uterus, through vessels, and into other bodily structures via a non-straight path. In another example, the tip portion 151 can comprise an antenna structure for propagating MW energy into body tissue. The electrode 150 is adapted to connect to the high-frequency system 100 by attaching electrode cable 154 to generator jack 116. The HF ablation system 100 comprises a HF generator of HF signal output, a control system with an master controller configured to control the ablation process, and a computer adapted to give a computer graphic display 101 of parameters of the ablation process. Connection 116 is adapted to carry HF power from the HF generator 100 through the electrode tip 151 to produce an ablation volume 194 within a target structure or structures 193 within the body 190. In some examples, target structure 193 can be a structure within an organ, such as a thyroid nodule (as shown in FIG. 1B), and in other examples the target can be a benign or malignant tumor or other tissue region, in the uterus, liver, lung, kidney, brain, nerve, bone, vertebra, thyroid, or another organ or other bodily region. As a schematic example in FIG. 1B, a target organ 193 can have a tumor within it, and the operator of the ablation apparatus desires the ablation volume 194 to cover, substantially cover, shrink, and/or destroy the tumor. It is understood that the neck and head of patient 190, thyroid gland 193, ablation zone 194, electrode 150, and other elements of FIG. 1B are not necessarily drawn to scale.

The probe active tip 151 includes a sharp tip point 151B, as well as echogenic markers 151A configured to produce an enhanced image of the active tip 151 when viewed using ultrasound imaging, as shown on ultrasound display 141. In one example, the echogenic marketers 151A can be depressions in the outer surface of the metallic active tip 151, arrayed around and along the active tip 151, having a substantially flat bottom in the wall of the active tip, and having isosceles triangular cross-section in a plane roughly parallel to the cylindrical outer surface of the tip 151, wherein one altitude of the triangular cross-section is parallel to the long axis of the tip 151 and shaft 152, and wherein the vertex of the triangular cross-section through which the altitude passes is closer to the distal point of the tip 151 than is the base side of the triangular cross-section to which the altitude is perpendicular. In this example, the two distal side faces of each triangular marker provide reflective surfaces for incoming ultrasound waves, the open space within each depression allows ultrasound waves access to the two distal side faces, and the triangular cross-section allows more markers to arranged longitudinally along the active tip relative to depression having a diamond- or square-shaped cross-section of the same width in the probe's circumferential direction. In one example, each of the echogenic markers 151A can have extent of 0.001-0.020 inches the longitudinal direction of tip 151, a 0.001-0.020 inches in circumferential direction of the tip 151, and depth of 0.001-0.006 inches in the tip wall (i.e. the tip radial direction). In some examples, the triangular cross-section flat-bottom depression can form a corner-cube reflector. In some examples, the echogenic markers 151A can take other forms, such as circular flat-bottom depressions, square flat-bottom depressions, diamond flat-bottom depressions, arbitrary triangular flat-bottom depressions, hemispherical depressions, corner-cube depressions, holes through the side of probe shaft 151, 152, roughing of the shaft surface, sand-blasting of the shaft surface, knurling of the surface, and combinations of these and other echogenic features in either identical or varied orientations. In some examples, echogenic markers 151A can be included in a metal surface under the electrical insulation of the insulated proximal shaft 152. In some embodiments, other arrangements of echogenic markers 151A can be included on probe 150 to highlight different features and dimensions of the probe 150. In some embodiments, the entirety of the shaft length 151, 152 can include echogenic features.

FIG. 1B shows schematically one example of a coolant supply system 130 to cool the active tip portion 151 of ablation probe 150. A reservoir 132 (such as a IV bag), in one example, contains water or saline cooled to a temperature less than body temperature, such as room temperature, approximately 20 deg C., near freezing, less than 10 deg C., or near 0 deg C. Tubing 155 carries the coolant through a peristaltic pump head 131 that pumps the coolant through the shaft of ablation probe 150. The electrode 150 has an internal channel through which coolant can flow to cool the probe active tip 151. The coolant can exit the probe 150 through tubing 156 and outflow into the collection reservoir 135. In some embodiments, the exit port 156A of the outflow tubing 156 is connected to the source reservoir 132, rather than a separate container 135 to provide for closed-loop cooling (an example of this is shown in FIG. 1D); for example, a needle can be connected to the exit end 156A of tube 156 and penetrated through a wall of reservoir 132, an opening can be cut in the wall of reservoir 132 and the outflow hole 156A of tube 156 can be inserted into the opening, the reservoir 132 can be configured with attachment ports matching the connectors of tubes 155 and 156, or another configuration. Preferably, as shown in FIG. 1D, the electrode-outflow tube 156 connects at an upper portion of reservoir 132, and the electrode-inflow tube 155 connects at a lower portion of reservoir 132 to provide for remixing of hotter water outflowing from probe 150 due to tissue heating. In some examples, reservoir 132 is cooled by external ice, a heat-pump, or another type of coolant system and so that the electrode coolant does not become too hot to effectively cool the electrode during a long ablation procedure. The pump 130 also includes a user control 133 to provide for manual control of pump function. The generator 100 provides an automatic check on the coolant flow into the electrode 150 and ablation electrode 150 when an ablation program is initiated by the user by holding the output level at a low level not expected to heat the electrode tip 151 even in the absence of cooling for an initial period 118C (visible on output level graph 102A in FIG. 1B), and only proceeding with delivery of ablation output if the electrode temperature, plotted by dotted line 102C, registers a value below a threshold indicative of proper electrode cooling, such as a temperature below 30 deg C. In some embodiments, the generator 100 does not proceed with ablation heating unless the temperature first registers a value indicative of body temperature, and then drops to a value indicative of coolant flow. Such temperature-dependent initiation of ablation output can have special advantage during cooled-electrode moving-shot ablation, thyroid ablation, and uterine fibroid ablation, wherein the fluid within electrode tubing 155, 156 can heat up while the electrode 150 is repositioned into between ablations at multiple positions, and thus it can be a logistical burden for the physician and staff to either (1) monitor the cooled electrode temperature before each initiation of ablation output, and/or (2) continuously run the pump 130, which has the disadvantages (a) that actively-flowing electrode coolant obscures monitoring of the post-ablation tissue temperature and thus a measure of ablation efficacy, (b) that coolant fluid heats up faster due to continuous circulation in relatively warmer surroundings, (c) that coolant fluid runs out fast in non-closed-loop coolant flow configurations (i.e. having a separate source container 132 and sink 135 container). In some embodiments, the generator 100 can discontinue the ablation program and signal an error condition to the user, if the coolant-check period 118C persists for longer than the typical time required for coolant to flow from the reservoir 132 to the electrode tip 151, for example 20-45 seconds depending on the pump rate. In some embodiments, the coolant check can be performed on an ongoing basis when ablation generator is being delivered to verify that the coolant flow is sufficient for the ablation output level delivered. In some embodiments, the generator 100 automatically activates the coolant pump via connection 134 when the user initiates an cooled-electrode ablation program, for example, by pressing the Start button 108; this has advantages for moving-shot ablation wherein the ablation output is repeatedly started and stopped in that it reduces attentional and procedural burdens for physician and staff, conserves coolant fluid volume by avoid unnecessary flow between ablations, reduces coolant fluid heat loss due to circulation through tubes and electrode residing in relatively hotter surroundings (i.e. the room, body, and ablation zone) and due to heat generated by the pump head's repeated mechanical and frictional interaction with the tubing, allows for electrode measurement of post-ablation tissue temperature (which would be obscured by the temperature of flowing coolant). In some embodiments, for example where connection 134 is absent, the user manually activates by control 133 the coolant pump to suit clinical needs.

Referring now to FIG. 1C, the generator 100 of FIG. 1B is shown in another mode of operation, namely non-cooled operation, in which pump 130 does not circulate coolant fluid to the probe 150. The value of generator setting 106E is changed to “Non-cool” from “Cooled” in FIG. 1B, so that the generator 100 does not activate pump 130 when the ablation output is activated. Time graphs for ablation output level 102A, impedance 102B, and probe tip temperature 102C display the history of the current non-cooled instance of RF output delivery. The output power 102A is automatically increased by the generator and then regulated such that that the temperature 102C of the ablation probe 150 is held at or near the target temperature setting value 106C, which is 90° C. in this example. The non-cooled probe 150 requires less power than power setting 106B to achieve the target temperature setting 106C. Corresponding to the power output variations 102A, the probe temperature 102C rises from the initial tissue temperature value up to the temperature setting value 106C. As the tissue heats, the impedance 102B first drops from its initial value, and rises modestly and slowly as the heated tissue 194A around the electrode tip coagulates, becoming more dense and drier. The digital readings for power 104B, impedance 104A, temperature 104D correspond to the rightmost vertical “y” value of the x-y plots for power 102A, impedance 102B, and temperature 102C, respectively, and the digital time reading 104C corresponds to the rightmost horizontal “x” value of those x-y plots. These plots 102A-C are plotted on the same time axis 102X. Readouts 103A and 103B display the portion of the current flowing to probe 150 that is flowing to each ground pad 121, 122. In other examples, between 1 and 4 ground pads 121 can be used at the same time, using jacks 115A-D, their currents being displayed by displays 103A-D. Display 104G displays the present amount of energy in kilojoule units delivered over this instance of RF output delivery; in other embodiments, energy can be reported in other units, such as calories, kilocalories, joules, and other units of energy. Display 104H displays the present average power in watts units delivered over this instance of RF output delivery. The “procedure total” area 104J displays the total energy delivered to tissue (“5.0 kJ” in this example), average power of ablation output delivery (“18.5 W” in this example), and total duration of ablation output delivery during all instances of ablation output delivery during the present medical procedure, including the instance of non-cooled RF output delivery depicted in FIG. 1C. In this example, these “procedure total” readings 104J account for the prior instance of cooled-electrode ablation output delivery depicted in FIG. 1B, which preceded the non-cooled-electrode output delivery depicted in FIG. 1C. Audio tone 107AA can be adapted to indicates temperature 104C, in addition or alternatively to impedance; this can be advantageous in non-cooled ablation, and all forms of temperature-controlled ablation, where probe temperature 104C is an important parameter for ablation control.

As shown by the size of ablation zone 194A (and its image on ultrasound display 141) in FIG. 1C relative to the size of ablation zone 194 (and its image on ultrasound display 141) in FIG. 1B, the ablation 194A produced by an ablation probe 150 when it is not cooled is typically smaller than the ablation 194 produced by the same ablation prove 150 when it is cooled.

Referring now to FIG. 1D, additional embodiments of the present invention are shown in a schematic scene in a medical-procedure room. The physician user 180 is performing moving shot RF ablation in the thyroid gland in the neck of patient 190 using generator 100 and ultrasound machine 140. Generator 100 includes an integrated pump 130 for fluid cooling of the ablation probe 150. Both hands of the physician 180 are occupied, the first hand holding the ablation probe 150, and the second hand holding the ultrasound transducer 145. The physician 180 observes an image 141 of the ablation probe 150 in body 190, listens to impedance-based audio signals 107AA from generator 100, moves the ablation probe 150, moves the transducer 145, activates and deactivates the generator's electrode output by means of footswitch 119. Intermittently the user 180 looks at the generator display 101 and manipulates the generator's controls 106G, 106F, 106E, 106EA, 106, 110A, 107A, 107B. Generator display 101 includes numeric readings for impedance 104A, power 104B, RF output time 104C, probe temperature 104D, and the duration of the most recent heating period 117A. Generator includes bar graphs 160A and 160B corresponding to the impedance 104A and power 104B readings, respectively. The bar graphs 160A, 160B are dynamic bar graphs whose top edges 161A, 161B move upward and downward corresponding to change in the readings 104A, 104B, respectively.

The impedance graph 160A includes a scale 163A by means of which user 180 can ascertain the value being graphed by the relative alignment of the top of the bar 161A and the labeled tick marks of the scale 163A. The value of the impedance graph 160A saturates at the top of the scale 163A in this example. Dotted line 162A indicates the minimum impedance measured during the current instance of RF output delivery; this has the advantage of helping the user 180 to determine the degree to which the present impedance 104A, 161A has risen relative to its minimum value; this can give the user a sense of how completely surrounded by boiling gas bubbles is the tip 151 of probe 150 and, thus, the progress of the ablation at current position of probe 150. The impedance bar 160A can be colored coded to match the impedance reading 104A in some embodiments. In some embodiments, the color of the impedance bar and/or the color of the impedance reading 104A can be coded to indicate the impedance value. For example, the impedance color can be green, when the impedance is in a low range indicative of non-boiling tissue and significant RF tissue heating; yellow, when the impedance is in a medium range indicative of a moderate degree of tissue boiling; red, when the impedance is a in a high range indicative of significant tissue boiling. For example, the impedance color can vary with the difference between the current impedance value and the most recent minimum impedance value. For example, the impedance color can vary with the difference between the current impedance value and the initial impedance when the generator's electrode output when last turned on by the user. Such color coding has the advantage of giving the physician user 180 an instant indication of the boiling and heating processes around the tip of the electrode 150 at a glance.

The audio signal 107AA is configured by settings 107A to indicate to the operator 180 the value of the impedance reading 104A and changes in the impedance reading relative to past impedance values. For example, the frequency of the audio signal 107AA can vary with the impedance reading, the audio signal 107AA when the impedance is a low range indicative of significant tissue heating can be distinctly audibly different from the audio signal 107AA when the impedance is in a high range indicative of tissue boiling and insignificant heating, or both. For example, the audio signal 107AA can produce an intermittent single beep (e.g. a single uninterrupted tone) when the impedance is in the low range, and an intermittent double beep (e.g. a sequence of two tone) when the impedance is in the high range, and the principle frequency of such beeps can vary with the measured impedance 104D (for example, the frequency can be a linear function of the impedance value). In some embodiments, the audible tone 107AA can provide both (a) an indication that the electrosurgical probe output of the generator 100 is active in accordance with certain standards for electrosurgical devices (e.g. IEC 60601-2-2 requires an audible signal having principal frequency in the range 100 Hz to 3 kHz when an electrosurgical output circuit is energized), and (b) an audible tone indicative of the impedance measurement; this can have the advantage of reducing the complexity of audible signals produced by generator 100 and improving the perception of the impedance tone 107AA by the user 180. The impedance-dependent audio signal 107AA has the advantage of providing the user 180 with information about the impedance measurement 104A of the generator 100, and thus the ablation process, without having to look at the generator display 101 while attending to the ultrasound image 141 and the patient 190 during the ablation process. This is an important advantage for moving-shot ablation, particularly in anatomical regions with a numerous sensitive structures (such as the thyroid gland or uterus), because the physician must monitoring ultrasound imaging 141 and generator impedance measurements 104A to safety and effectively control the ablation process at multiple locations, possibly moving the ablation probe 150 while the generator's ablation-probe output is active. In some embodiments, the audible signal 107AA can indicate RF output power and can audibly indicate to the user 180 if the power output is in a range capable of significantly expanding the ablation zone, or in a range not capable of significantly expanding the ablation zone.

The power graph 160B includes a scale 163B by means of which user 180 can ascertain the value being graphed by the relative alignment of the top of the bar 161B and the labeled tick marks of the scale 163B. The scale 163B can vary with the target power value 106B, for example, upper limit of the power scale 163B can be set modestly higher than the target power setting 106B so that the measured power output reading is visible on the scale and its variations can be appreciated by the user by means of variations in the power graph 160B height. Dotted line 162B indicates the average power output value during the current instance of RF output delivery; this has the advantage of helping the user 180 to determine the degree to which the output power is achieving its target value for the entirety of the ablation session, rather than just instantaneously as is conveyed by the present power reading 104B, 161B. The power bar 160B can be colored coded to match the power reading 104B in some embodiments. In some embodiments, the color of the power bar 160B and/or the color of the power reading 104B can be coded to indicate the power value. For example, the color can indicate whether or not the power output is in a range capable of heating the tissue to a destructive level and/or efficiently expanding the size of the ablation zone (both examples of “sufficient” or “significant” tissue heating). Such color coding has the advantage of giving the physician user 180 an instant indication of the boiling and heating processes around the tip of the electrode 150 at a glance.

The embodiments described in relation to bar graphs 160A, 160B can be applied to bar graphs for other generator readings, including, without limitation, temperature, current, voltage, power, and impedance. In some embodiments, the dotted lines 162A, 162B can indicate other statistics of a graphed reading and its history, such as a minimum maximum, average, median, or mode. In some embodiments, multiple statistic lines 162A, 162B can be included on the single bar graph 160A, 160B. In some embodiments, a bar graph can be either vertically oriented, horizontally oriented, or oriented in another direction. In FIG. 1D, the bar graphs 160A, 160B are vertically oriented. The embodiments described in relation to bar graphs 160A, 160B can be applied to other dynamic graphs, for example, having shapes other than a bar, such as circular graph whose radius plots a generator reading, for which a circle indicates a statistic of the reading and its history.

Continuing to refer to FIG. 1D, the embodiment of generator 100 includes user-interface controls to start the ablation-probe output 108, stop the ablation-probe output 109, select RF ablation output 106G, select nerve-stimulation output 106F, select ablation probe cooling 106E, select non-cooling of the ablation probe 106EA, adjust generator settings 106, increase (+) and decrease (−) the output level 110A, adjust the audio signal 107A, edit and access generator procedure records 107B. Ultrasound machine 140 displays ultrasound image 141, which, in this embodiment, shows the ablation probe 150 and surrounding anatomy of the neck of patient 190. For example, the dotted curve depicts superficial tissue layers such as skin and muscle and capsule of the thyroid gland, the dashed ellipse can depict a thyroid nodule, and the small solid-lined ovals can depict formation of gas bubbles early in an ablation heating process around the tip of probe 150 wherein the bubbles do not yet densely surround the tip, indicating the user that the tissue is indeed heating sufficiently to cause some focal boiling but not so much that the ablation zone has stopped growing due to gas bubbles fully preventing the flow of RF electrical current to the tissue. Such partial gas formation typically corresponds to impedance values that are roughly equal to the impedance value before heating (being either lower or higher depending on the particular timing and characteristics of the ablation process) and that have not yet markedly increased from the impedance value before heating as initiated. The correspondence between indications of tissue heating and boiling on the ultrasound machine 140 and generator 100 are not one-to-one; that is, the ultrasound 140 and generator 100 provide complementary information about the tissue 190 and ablation process. For example, a very high impedance measurement, or markedly lowered power measurement for high applied voltage, by an RF generator 100 can quantitatively indicate that an ablation process is compete at the current probe 150 location, whereas a dense cloud of bubbles around the tip of the probe 150 on ultrasound image 141 provides a non-quantitate indication of ablation size, but may not show if there are any low-impedance pathways between the bubbles through which additional applied RF current can materially increase ablation size at the present location of probe 150. Similarly, when RF energy and pump cooling are not being applied to the probe 150, the probe temperature 104D measured by the generator can provide a numeric indication of the treatment or non-treatment of tissue at a particular location by thermal ablation (and the possible need for continued ablation energy delivery at that location), whereas the ultrasound image can provide a broad, but non-quantitative, indication of the treatment or non-treatment of tissue (e.g. treatment being indicated by a low intensity region within which there are smaller high intensity regions). As such, it is advantageous that a physician user 180 can simultaneously attend to ultrasound 140 and generator 100 data during an ablation process, particularly a moving-shot ablation process in the thyroid, uterus, or other organs.

Ultrasound machine 140 includes ultrasound controls 142, as well as controls 143 for wireless control of the generator 100. The generator 100 can also include settings 106 for wireless control of the ultrasound machine, as well as wireless (or in some embodiments, wired) data sharing, display of generator data on the ultrasound machine, display of ultrasound data on the generator screen, or display of both generator and ultrasound data on third screen. Such data sharing and display of generator and ultrasound data on a single screen has the advantage for moving-shot ablation in organs such as the thyroid and uterus and other organs that the user 180 can more easily keep the user's attention on both data sources at the same time while the ablation probe 150 is being moved and activated in patient tissue 190.

Continuing to refer to FIG. 1D, Probe 150 is connected to the electrode-output of generator 100 by cable 154. Pump head 130 pumps fluid coolant from IV bag 132 via tubing 155 to probe 150. Fluid coolant returns to bag 132 via tubing 156 whose exit end 156A in inserted into the top of bag 132 (for example by cutting a hole in bag 132 or penetrating the bag with a needle attached to the tubing exit end 156A) to create a closed-loop fluid-cooling system for probe 150, which is inserted percutaneously into a thyroid nodule within the thyroid gland within the neck of patient 180. Ground pads 121, 122 are placed on the thighs of patient 180 and are respectively connected to generator 100 by cables 121A, 122A to carry return current from probe 150. Patients 190 lies on a procedure couch and is covered by a drape. Portions of the patient 190 under the drape are drawn with dashed outlines. The ground pads 121, 122 and portions of their cables 121A, 122A that are under the drape are drawn with dotted lines. Ultrasound transducer 145 is applied to the skin of neck of patient 180 to scan probe 150 and surrounding anatomy, an image of that scan being displayed on ultrasound monitor 141 of ultrasound apparatus 140. Transducer 145 is connected to ultrasound apparatus 140 via cable 145A. Cable 119D connects footswitch 119 to generator 100; it is understood that the drawing of cable 119A is schematic and, in a more realistic scenario, such a cable 119A would run under the couch to connect to generator 100 on a shorter, more realistic path.

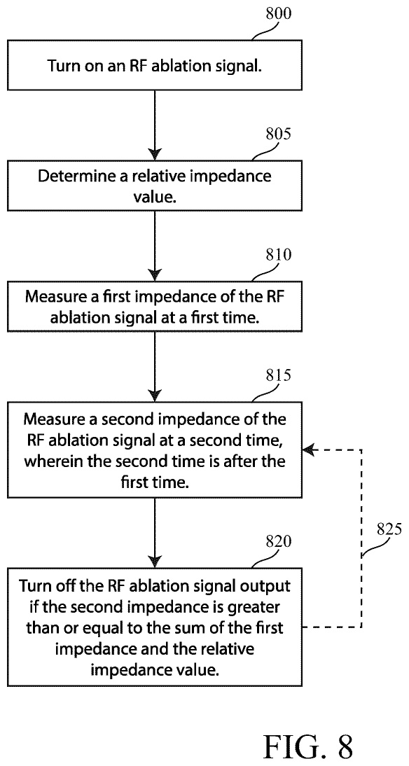

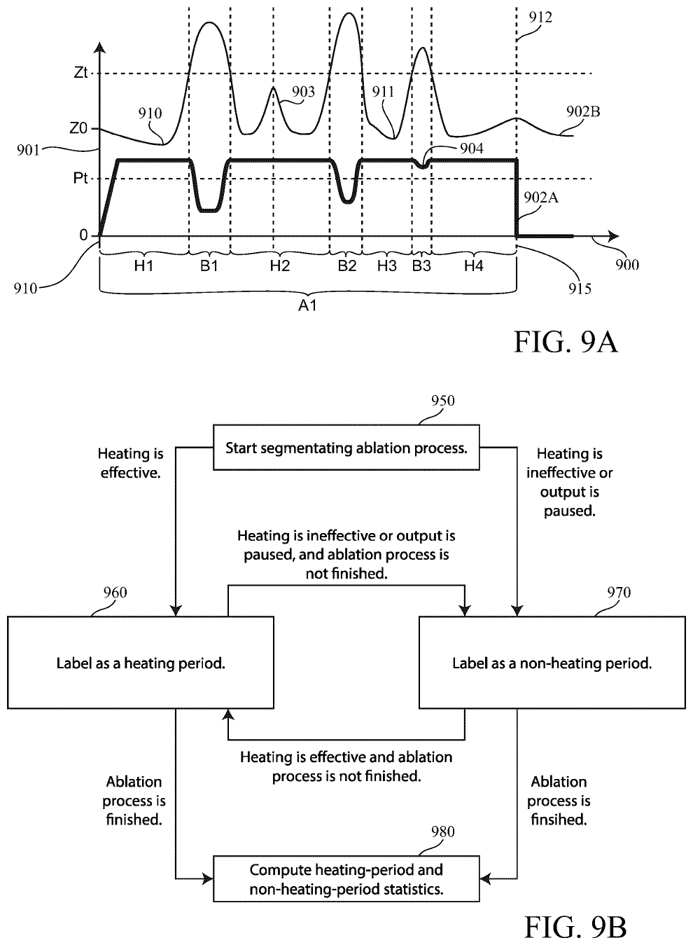

The impedance-based audio feedback 107AA; graphs 160A, 160B with historical reading statistics 162A, 162B; footswitch 119; automated coordination of the outputs of the generator 100 and pump 130; automatic utilization of electrode temperature to identify proper electrode cooling; automatic storage of generator readings to an persistent record (e.g. electronic data file); automatic computation of statistics and readings that characterize the current instance of electrode output delivery 162A, 162B, 117A, 104G, 104H; generator cut-off controls based on relative impedance values (see FIG. 8); and statistics and readings that characterize the current ablation procedure as a whole (104J, and heating and non-heating statistics described in relation to FIGS. 9A and 9B) individually and collectively allow the physician user 180 to focus attention on the ultrasound image 141 and patient 190 during moving shot ablation, such as thyroid ablation, because they reduce the need for the physician 180 or physician's staff to regularly monitor and record other data, remember and perform computations with multiple past values of such data, regularly use each hand for more than one operation, and perform other actions. Time graphs, such as 102A, 102B, 102C in FIG. 1B provide an even richer persistent depiction of historical readings that the user 180 can intermittently look at to assess the ablation process, while otherwise focusing attention on the ultrasound image 141 and patient 190. These are advantages of the generator 100, system, and methods that are part of the present invention.

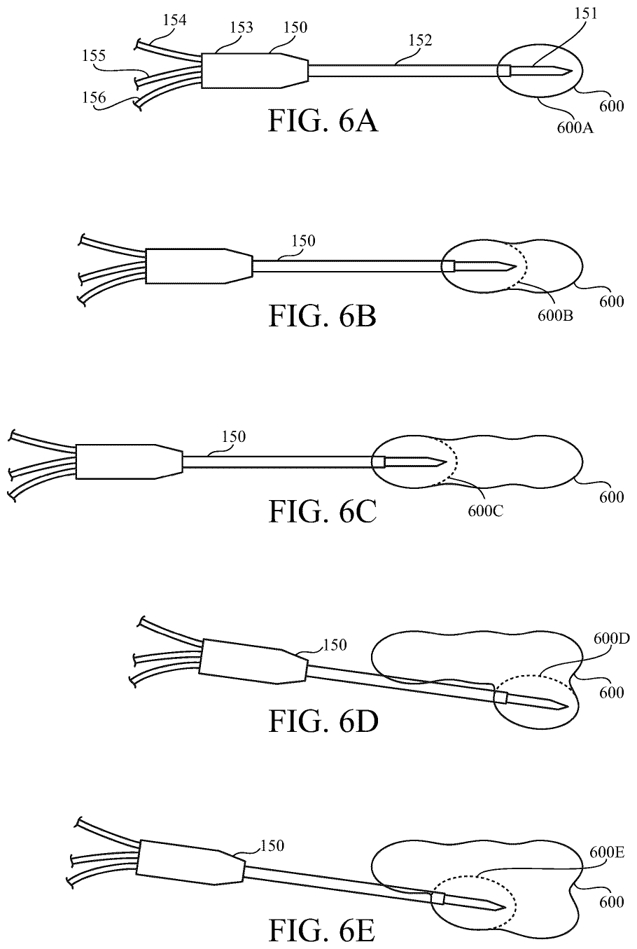

Referring now to FIGS. 6A, 6B, 6C, 6D, and 6E, an example of a moving-shot ablation process is shown schematically. Ablation probe 150 includes generator cable 154 (an abbreviated portion of which is shown), coolant inflow tube 155 (an abbreviated portion of which is shown), coolant outflow tube 156 (an abbreviated portion of which is shown), handle 153 at the electrode proximal end, and shaft 151, 152 at the electrode distal end, wherein the ablation zone forms in tissue predominantly around the distal active tip 151 (which is an electrically-conductive portion from which RF current flows in embodiments where probe 150 is an RF electrode), wherein the ablation zone does not substantially form around tissue around proximal shaft portion 152 (which is an electrically-insulated portion from which RF current does not substantially flow in embodiments where probe 150 is an RF electrode). In FIG. 6A, a first portion 600A of aggregate ablation zone 600 is generated around the probe tip 151 (by delivery of RF output via active tip 151 for embodiments in which probe 150 is an RF electrode). The probe 150 is then withdrawn longitudinally (i.e. in the direction of the probe's shaft length 151, 152), with the ablation output either turned off or not turned off, by approximately the length of active tip 151 to reach the position of probe 150 that is shown in FIG. 6B. Then, a second portion 600B of the total ablation zone 600 is generated around the active tip 151 in that position. The ablation zones 600A and 600B are blended together by thermal diffusion within the tissue to form total ablation zone 600, and an inner border of zone 600B is depicted by a dotted outline within total ablation 600. The probe 150 is then again withdrawn longitudinally by approximately the length of active tip 151, with the ablation output either turned off or not turned off, to the position show in FIG. 6C. In that position, ablation zone 600C is generated around tip 151 to further expand the total ablation zone 600. A border of zone 600C is depicted within total zone 600 by means of a dotted outline. The ablation output delivered via probe 150 is then stopped and then probe is redirected and advanced to a location to the side of the current elongated ablation zone 600, as shown in FIG. 6D. Ablation zone portion 600D is then generated around the tip 151 at that position, thereby expanding the total ablation zone 600 lateral to the ablation zone 600 depicted in FIG. 6C. A border of zone 600D interior to zone 600 is shown in a dotted outline. The probe 150 is then withdrawn longitudinally by a distance that is less than or equal to the tip length 151 to the position shown in FIG. 6E. At this location, ablation portion 600E is generated, further expanding the total ablation zone 600. A border of zone 600E interior to zone 600 is shown in a dotted outline. In some embodiments, this process can be continued to further expand zone 600 is a fan-like shape. In some embodiments, the ablation probe 150 can be redirected in another direction out of the plane of the FIGS. 6A-E in order that the total ablation zone is expanded in all three dimensions. In this example, incremental ablation zones 600A-600E are approximately ellipsoidal in shape.

Referring now to FIG. 2, in one embodiment, this invention relates to a systems and methods that include stimulating nerves before thyroid ablation by means of an electrical nerve stimulation signal delivered through the ablation probe 150. The ablation probe 150 can be an RF electrode, a microwave ablation antenna, an IRE electrode, an electrosurgical probe, another type of ablation probe. Such a nerve stimulation signal, in one example, can be a biphasic square wave pulse that is delivered intermittently or periodically. For example, as shown in FIG. 2A, waveform 200 includes three cycles of such a nerve stimulation signal, including three biphasic pulses 205, 210, 215. Such a biphasic square wave pulse can have positive voltage +V for duration w/2 milliseconds, followed by a negative voltage −V of duration w/2 milliseconds. The voltage can be a value selected from the list: 0-10 volts, a value greater than 10 volts, a value known to those skilled in the art of nerve stimulation. The duration w can be a value selected from the list: 0.5, 1, 2, 5, 10 milliseconds (msec); a value in the range 0-10 msec; a value higher than 10 msec; a value known to those skilled in the art of nerve stimulation. Such a biphasic square wave pulse can be delivered with frequency f, where f can be selected from the list: 1, 2, 3, 4, 5, 6, 7, 8, 9, 10, 11, 12, 13, 14, 15, 20, 50, 80, 100, 150, 200 Hz; a positive value in the range 0-200 Hz; a value higher than 200 Hz; a value configured to evoke muscle contractions in the larynx; a value configured to evoke signal measurable by electromyography (EMG), for example by means of electrodes placed to contact or to be in proximity to the vocal cords; a value configured to evoke patient sensation of vibration in their vocal chords and/or larynx; a value in the configured to evoke visible muscle contractions, such as value in the range 0-5 Hz; a value to known to one skilled in the art of laryngeal nerve stimulation. In some embodiments, the frequency can be fixed such that the signal is substantially periodic. In some embodiments, the frequency can be an average value such that inter-pulse interval varies to some degree. The amplitude V, duration w, frequency f of such a biphasic square wave pulse train can be configured to evoke a detectable response when applied to a nerve, such as the recurrent laryngeal nerve, superior laryngeal nerve, or vagus nerve. Note that, typically, waveform 200 would include many more pulses than shown in FIG. 2a, and the overall duration of the waveform can last up to a minute or more, depending, for example, on how long the physician spends ascertaining physiological responses to such a signal. In the example shown in FIG. 2A, the third pulse 215 has a different amplitude V2 than the first two pulses V. The amplitude of pulses in a nerve simulation waveform can vary, for example, as the amplitude is ramped up to a desired value at the beginning of delivery of the signal, or as the amplitude is varied by the physician as the physician determines physiologic responses to the signal. In some embodiments, the stimulation amplitude can ramp up automatically to a set value in accordance with a user setting, and in some embodiments, the stimulation amplitude can be adjusted manually by the physician 180.

In some embodiments, the amplitude V of such a biphasic square wave pulse (e.g. waveform 200) can be regulated to control its electric current, meaning that the pulse amplitude V can be an electric current value, rather than a voltage value. The current value can be a value selected from the list: 0-20 mA, a value greater than 20 mA, a value known to those skilled in the art of nerve stimulation, a value configured to have a transient and/or non-destructive effect when applied to nerves and other structure in and around the thyroid gland. One advantage of a current-regulated square biphasic pulse is it substantially charge-balanced, meaning that the pulse tends not to lead to a net accumulation of charge within the tissue. In some embodiments, the pulses of a current-controlled nerve stimulation signal can take other shapes over time, the shape being a time-varying value of electric current, for example, a monophasic square pulse, a monophasic non-square pulse, a biphasic non-square pulse, a monophasic pulse, a biphasic pulse, a charge-balanced pulse, or another pulse shape known to one skilled in the art of nerve stimulation.

In some embodiments, the nerve stimulation signal can take other forms such as a monophasic pulse series, or intermittent or periodic pulses of other signal shapes and symmetries, or pulses for which electrical parameters other than voltage and current are regulated.

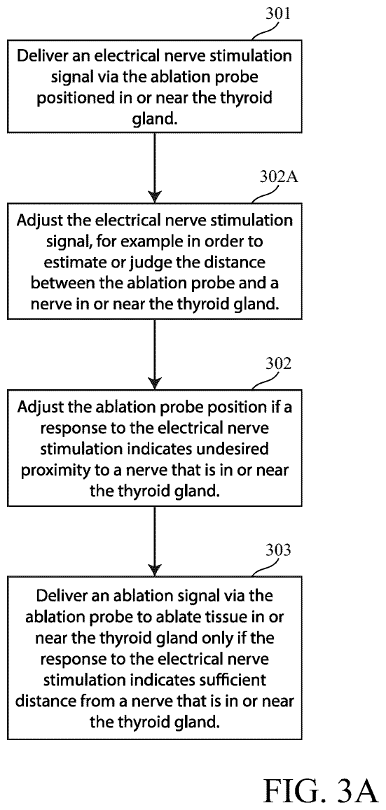

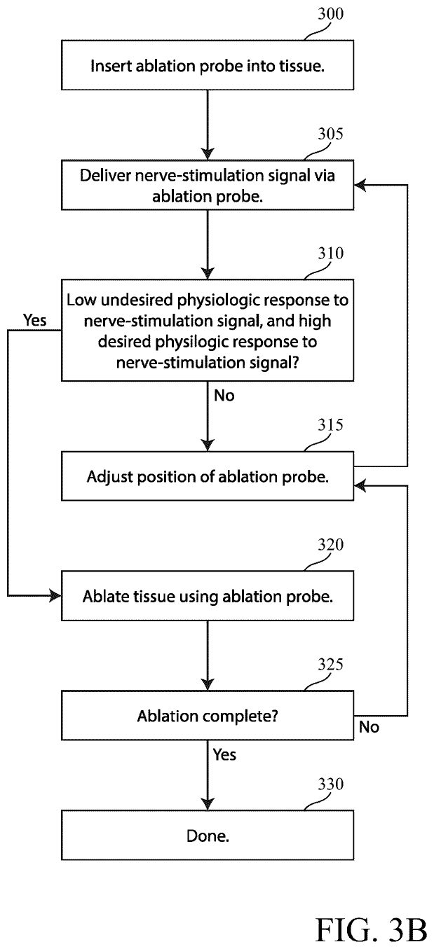

Referring now to FIG. 3A, in one embodiment, the method of nerve stimulation and RFA includes the steps:
1 Deliver an electrical nerve stimulation signal via the ablation probe positioned in or near the thyroid gland (step 301).
2. Adjust the ablation probe position if a response to the electrical nerve stimulation indicates undesired proximity to a nerve that is in or near the thyroid gland (step 302).
3. Deliver an ablation signal via the ablation probe to ablate tissue in or near the thyroid gland only if the response to the electrical nerve stimulation indicates sufficient distance from a nerve that is in or near the thyroid gland (step 303).

Note that “proximity” and “distance” in steps 302 and 303 refer to the proximity and distance of the ablation probe to a nerve. “Undesired proximity” can entail that the ablation zone produced by the ablation probe is unacceptably likely to damage a nerve. “Sufficient distance” between the ablation probe and a nerve can mean that the nerve is unlikely to be damaged by the ablation zone produced by the ablation probe. Step 2 of the above method can further include the step 302A of adjusting the electrical nerve stimulation signal, for example, adjusting the amplitude V of the signal, in order to estimate or judge the distance between the ablation probe and a nerve in or near the thyroid gland.

In some embodiments, the above method (i.e. FIG. 3A) can be limited to pertain only to an ablation probe positioned in the thyroid gland.

In some embodiments, the above method (i.e. FIG. 3A) can be limited to pertain only to an RF electrode and RF ablation signal.

In some embodiments, the response to the electrical nerve stimulation (such as by a signal such as 200) can be a voice change in, evoked muscle stimulation in, or sensation experienced by the person 190 in whose thyroid 193 the RF electrode is positioned (the “patient”). In some embodiments, the method can include asking patient 190 to speak during nerve stimulation and assessing voice change. In some embodiments, the method can include feeling for muscle stimulation at the skin surface 191 of the patient 190, for example, at the skin overlaying the thyroid gland 193, larynx, or the neck. In some embodiments, the method can include asking the patient 190 to describe their sensations, such as a sensation of vibration in their voice box, as nerve stimulation is applied. In some embodiments, the method can include setting a threshold for the amplitude V of the nerve stimulation signal in relation to the response, for example, a threshold below which the electrode is considered to be too close to a nerve to safety perform tissue ablation. For example, if a response to the nerve stimulation signal is detected when the signal's amplitude is less than a threshold, then the electrode is too close to a nerve to perform ablation safely, and the electrode should be moved. For example, the method can include a maximum stimulation voltage V at which muscle stimulation can be felt, stimulation sensation can be felt by the patient, or voice is affected before ablation is performed.

Referring now to FIG. 2B, in some embodiments of the nerve-stimulation methods of FIGS. 3A and 3B, and of associated systems, the nerve stimulation signal and ablation signal can be delivered at the same time to provide for (a) continuous impedance monitoring, and/or (b) simultaneous nerve stimulation and ablation of tissue. Signal 250 (shown as a solid line) is a schematic example of such a signal, which is the superposition (also known as “addition”, “composition”, “simultaneous delivery”, “delivery at the same time”) of an RF sinusoidal signal and nerve-stimulation signal that consists of periodic, biphasic square pulses. The nerve-stimulation component 252 of signal 250 is shown as a dotted outline. It is understood that RF signals can have principal frequency f_RF in the range 100,000 to 1,000,000 Hz (or another frequency known to one skilled in the art of RF ablation), and thus have a period 1/f_RF in the range 0.000001 to 0.00001 seconds that is substantially shorter than the period 1/f of a nerve stimulation signal; the sinusoidal, biphasic pulse train, and other elements of signal 250 in FIG. 2B are not necessarily drawn to scale. Three nerve-stimulation pulses 255, 260, 265 of signal 250 are shown in FIG. 2B, each having pulse width w, the first two pulses having amplitude V, the third pulse having a different amplitude, and the pulses being delivered with frequency f (which corresponds to period 1/f). The RF signal component of signal 250 has amplitude A, such that the total amplitude of signal 250 is A in between pulses, the maximum value of signal 250 is V+A during pulse 255 and pulse 260, and the minimum value of signal 250 is −(V+A) during pulse 255 and pulse 260. Just as for signal 200, a typical nerve simulation signal 250 would, in practice, typically include more nerve stimulation pulses than shown in FIG. 2B. In some embodiments, the stimulation signal 250 can be applied both when the ablation signal component is configured to ablate tissue (e.g. having a high amplitude A), and when ablation signal component is configured not to substantially heat tissue (e.g. have a low amplitude A). In some embodiments, the nerve stimulation level of signal 250 can be set to a level V such that a response to the electrical nerve stimulation indicates undesired proximity to a nerve, so that the physician gets an indication of undesired proximity to nerves throughout an a process of ablating tissue at multiple tissue locations, including while the electrode is being moved and the ablation signal component is adapted to substantially ablate tissue, and/or including while the electrode is being moved and the ablation signal component is adapted to not substantially ablate tissue. These embodiments have a special advantage to simplify ablation during moving-shot thyroid ablation because the electrode is moved to ablate tissue at multiple locations (perhaps number up to 5, 10, 20, 30, 40, 50 60, or more locations) during a single treatment, such that alternating between stimulation and ablation would add substantial procedure complexity. In some embodiments, the method of FIG. 3A can be applied in and around anatomical structures other than the thyroid gland. In the case of an RF ablation signal, both the nerve stimulation signal and the RF ablation signal are delivered to tissue via electrical conduction, and as such, it is an advantage that this method be applied to RF ablation because both the stimulation and ablation signals can be delivered via the same electrode contact (“active tip” 151).

In some embodiments of the above method (i.e. FIG. 3A), the ablation signal is applied after the nerve stimulation is applied, as indicated in the numbered steps above.

In some embodiments of the above method (i.e. FIG. 3A), the number steps above are repeated, for example as the ablation probe is moved to multiple positions in the tissue.

One advantage of these embodiments (e.g. those described in relation to FIGS. 2A, 2B, 2C, 2D, 3A, and 3B) is that damage to nerves in and near the thyroid gland due to RFA can be limited.

Referring now to FIG. 3B, a method is presented in which a nerve-stimulation signal is applied to tissue via an ablation probe 150. In step 300, an ablation probe 150 is inserted into patient tissue 190, for example, tissue in or around the thyroid gland, uterus, or another organ. In the following step 305, a nerve-stimulation signal (such as signal 200) is applied to the ablation probe 150, for example, by generator 100 and/or by means of the circuits of FIG. 2C or FIG. 2D. In step 305, the amplitude, frequency, or other parameters of the nerve-stimulation signal can be adjusted. In the following step 310, the physiologic response of the patient tissue 190 to the nerve-stimulation signal is assessed. Physiologic responses can include voice changes in the patient 190, muscle contractions in the patient 190, sensations perceived by the patient 190, pain, heart fluttering, heart rate changes, and physiologic effects on the patient 190 that may be related to nervous activity. Such responses can include undesired physiologic responses that indicate undesired proximity of the ablation probe to a nerve, and desired physiologic responses that indicate desired proximity of the ablation probe to a nerve. If there is a low level of undesired physiologic responses (e.g. an acceptably low or vanishing amount or intensity of an undesirable physiologic response for a given level of nerve-stimulation signal intensity) and a high level of desired physiological responses (e.g. an acceptably high or above-threshold amount or intensity of a desirable physiologic response for a given level of nerve-stimulation signal intensity), then the next step after step 310 is step 320 (i.e. the method follows the “Yes” arrow). Otherwise (i.e. there is unacceptable degree of undesirable physiologic response, or an unacceptable degree of desirable physiologic response), the next step after step 310 is step 315 (i.e. the method follows the “No” arrow). Note that, in some embodiments, step 310 omits criteria for undesired physiologic responses, so that nerve stimulation is only directed toward moving the ablation probe sufficiently close to nerve locations where ablation is desired. Also note that, in some embodiments, step 310 omits criteria for desired physiologic response, so that nerve stimulation is only directed toward moving the ablation probe sufficiently far away from nerve locations where ablation is not desired. In step 315, the position of the ablation probe is adjusted in hope of changing the distance of the ablation probe to certain nerves. After step 315, step 305 (i.e. nerve stimulation) is repeated. If step 320 is reached, then the ablation probe is presumed to be in an acceptable location relative to nerves, and tissue near the ablation probe 150 is ablated, for example, by the delivery of destructive RF energy to the tissue by the ablation probe 150. After step 320, in step 325, the completeness of the ablation process is assessed. If the ablation is not complete, for example if there are other locations to be ablated in a moving shot ablation process, then the next step after step 325 is step 315 (i.e. the method follows the “No” arrow), and that step 315 and certain other steps are repeated as described above. However, if the ablation is complete, for example if all anatomical structures appear to have been destroyed by ablation, then the next step after step 325 is step 330 (i.e. the method follows the “Yes” arrow) and the method is complete. In some embodiments of FIG. 3B, the ablation probe is configured to ablate tissue throughout all steps; in such embodiments, the nerve-stimulation is applied as the active ablation probe is moved around within the tissue, and movement of the ablation probe is paused in step 320 in order to enlarge the ablation zone at a location in the tissue.

In some embodiments, the nerve stimulation signal can, additionally or alternatively, be applied to an ablation probe 150 in order to move the ablation probe closer to a target nerve in order to ablate the nerve to have a clinical effect, such as pain relief or other effect of denervation. In such embodiments, the position of the ablation probe is adjusted until a desired response, such as reproduction of pain or muscle twitching, is detected at a sufficiently low nerve-stimulation signal amplitude V.

In some embodiments, the RF generator 100, RF electrode 150, and RF signal can be replaced by an IRE generator, IRE electrode, and IRE signal; an MW generator, MW probe, and MW signal; or another type of ablation generator, electrode, and signal. In the example of a MW generator and MW probe, the MW probe can include an electrically-conductive contact for conduction of the nerve-stimulation signal to tissue.

In some embodiments, the generator 100, ablation probe 150, and electrical ablation signal (such as the RF component of signal 250, or the signal that generated by RF source 280) can be replaced by a generator, ablation probe, and non-electrical means of ablation, wherein the ablation generator can produce a nerve-stimulation signal and the ablation probe includes an electrical contact for delivery of the nerve-stimulation signal. For example, a cryoablation apparatus can include a nerve-stimulation signal generator, a cryoablation probe can include an electrical contact for conduction of the nerve-stimulation signal to tissue, and the cryoablation apparatus can deliver cryogenic fluid or gas to the cryoablation probe for cryoablation of tissue. For example, a laser-ablation generator can include a nerve-stimulation signal generator, a laser-fiber or its introducer can include an electrical contact for conduction of the nerve-stimulation signal to tissue, and the laser-ablation generator can deliver laser light to tissue via the laser-ablation fiber to the cryoablation probe for cryoablation of tissue.

In another embodiment, the present invention pertains to an RF generator 100 that is configured for moving-shot ablation, and/or in particular for moving-shot thyroid ablation, wherein the generator 100 further includes a nerve stimulator that can deliver an electrical nerve stimulation signal to an RF electrode 150. One advantage of this embodiment is that damage to nerves in and near the thyroid gland due to RFA can be reduced in likelihood and/or degree. In some more-specific embodiments, the RF generator is configured to deliver an RF signal and a nerve stimulation signal simultaneously (e.g. signal 250), non-simultaneously (e.g. signal 200), or both, in different modes of operation. In some more-specific embodiments, the RF generator 100 is configured to deliver the nerve stimulation signal at the same time as an RF signal configured to substantially heat tissue, or at the same time as an RF signal configured not to substantially heat tissue (but perhaps only to monitor tissue impedance), or both (for example, see waveform 250). One advantage of these embodiments is that a nerve stimulation signal (such as signals 200 and 250) can be applied to judge undesired proximity to nerves whose ablation could lead to functional deficits or other damage. One advantage of a system that can deliver a nerve ablation signal at the same time as an RF signal that is configured to heat tissue (e.g. waveform 250), is that it can substantially reduce the complexity of moving-shot RF ablation by providing for nerve stimulation while the electrode is moving to ablate multiple tissue locations during a treatment. This is a special advantage of thyroid moving-shot ablation because such treatments can involve ablation at up to 60 or more nearby locations, such that interleaving stimulation and RF ablation could make use of nerve ablation at every location cumbersome.

FIG. 2C shows one example of an electrode-output circuit for an RF generator 100 that can produce both an RF ablation signal and a nerve stimulation signal. In this example, the RF supply 280 (which can be a voltage, current, or power source in some embodiments) and nerve-stimulation supply 290 (which can be a voltage or current supply in some embodiments) each be individually activated (at the same time or at different times) by the generator controller in accordance with user settings, such as 106G and 106F, respectively. RF supply 280 generates an RF ablation signal, e.g. a sinusoidal electrical waveform with principle frequency in the range 100,000 to 1,000,000 Hz. Nerve-stimulation supply 290 generates a nerve stimulation signal, such as signal 200. The RF ablation source and nerve-stimulation source are connected in series in this schematic circuit. The voltage signals produced by supplies 280 and 290 sum to generate a voltage signal between output poles 285 and 286. In some embodiments, pole 286 can be an output connection for electrode 150, such as jack 116 in FIG. 1B, and pole 285 can be connected to one or more output connections for ground pads 121, 122 (also known as “neutral electrodes”), such as jacks 115A, 115B, 115C, 115D in FIG. 1B. In embodiments where electrode 150 is a bipolar electrode, poles 285 and 286 each connect to a different electrical contact on the shaft of electrode 150 via cable 154. In embodiments for “dual” RF (also known as, inter-probe bipolar RF, wherein RF current flows between two ablation probes), poles 285 and 286 are each connected to the electrical contact on a different ablation probe. In FIG. 2C, closing switch 281 will short supply 280 out of the circuit, effectively disabling its output, and opening switch 281 includes supply 280 in the circuit. In FIG. 2C, closing switch 291 will short supply 290 out of the circuit, effectively disabling its output, and opening switch 291 includes supply 290 in the circuit. Activating only supply 280 applies an RF output signal to the output poles 285, 286. Activating only supply 290 applies a nerve-stimulation signal (such as signal 200) to the output poles 285, 286. Activating both supplies 280, 290 at the same time applies a combined RF and nerve-simulation output signal (such as signal 250 in FIG. 2B) to the output poles 285, 286.

FIG. 2D shows another example of an electrode-output circuit for an RF generator 100 that can produce both an RF ablation signal and a nerve-stimulation signal. The same circuit components from FIG. 2C are rearranged such that the RF supply 280 and nerve-stimulation supply 290 are arranged in parallel to produce an output signal across poles 285, 286. In some embodiments, supplies 280 and 290 can be current sources such that their output currents add. In FIG. 2D, closing switch 281 includes the output of supply 280 in the circuit of the circuit, and opening switch 281 disconnects supply 280 from the circuit, effectively disabling its output. In FIG. 2D, closing switch 291 includes the output of supply 290 in the circuit of the circuit, and opening switch 291 disconnects supply 290 from the circuit, effectively disabling its output.

In another embodiment, the present invention pertains to a system that includes an RF generator 100 and an electrode 150, wherein the RF generator 100 is configured to deliver an RF signal to the electrode and thereby ablate tissue that is in contact with the electrode; wherein the RF generator is configured to deliver a second electrical signal (such as signal 200) to the electrode that is configured to stimulate nerves in and around tissue that is in contact with the electrode; wherein the generator is configured to control the amplitude the RF signal (such as amplitude A in FIG. 2B); wherein the generator is configured to prevent cut off the RF signal when the impedance of the signal's circuit (such as that displayed by display 104A) rises. In some embodiments, such prevention of cut off can be omission of an open-circuit signal cut-off control. In some embodiments, such prevention of cut off can be a limit on, or reduction of, the RF signal amplitude as the impedance increases, such that the impedance will tend not to exceed the generator's high-impedance cut-off threshold even with continued delivery of RF signal output. In some embodiments, the limit can be a limit of the maximum voltage of the RF signal (such as a limit on the voltage of amplitude A in FIG. 2B). In some embodiments, the limit can be a limit on the power of the RF signal that decreases as impedance increases. In some embodiments, the amplitude of the RF signal can be a characteristic selected from the list: voltage, current, power. In some embodiments, the system can further include a fluid pump 130 that delivers fluid to the shaft of the electrode 150. In some embodiments, the system further includes a fluid pump 130 for fluid cooling of the electrode 150. One advantage of the system is that nerve simulation can be performed during a moving-shot RFA procedure in the thyroid gland to help avoid damage to nerves in or near the thyroid gland, such as the recurrent laryngeal nerve, superior laryngeal nerve, and branches thereof. In some embodiments, the RF generator can deliver the first signal and the second signal at the same time.

In another embodiment of the present invention, a non-cooled RF electrode 150 is used for ablation in the thyroid gland (for example, as shown in FIG. 1C). One advantage of this embodiment is that the ablation zone (volume of tissue heated to a destructive level) produced by a non-cooled electrode 150 tends to be smaller than that produced when an electrode is fluid-cooled, and thus the risk of inadvertent damage to nerves and/or other sensitive structure in and around the thyroid gland (such as the trachea, blood vessels, skin, muscles; see FIG. 1A) can be reduced. Another advantage of such an embodiment is that the non-cooled RF electrode 150 can include a temperature sensor that measures tissue temperature and thus provides an additional piece of information about tissue heating and the ablation process.

In a more particular embodiment, the temperature measured at the active tip 151 of a non-cooled electrode 150 (i.e. portion of the electrode through which RF energy is delivered to the tissue in order to heat the tissue) is controlled, an example of which is depicted by temperature graph 102C in FIG. 1C. In particular, the temperature can be controlled be to less than boiling (i.e. less than approximately 100° C.), thereby avoiding unpredictable irregularity in the ablation zone, which can be produced intentionally or unintentionally when temperature is not controlled (as is typically the case for cooled RF electrodes). One advantage of this embodiment, is that the size of the ablation zone can be controlled precisely, reproducibly, and/or gradually in order to limit inadvertent damage to nerves and/or other sensitive structure in and around the thyroid gland (such as the trachea, blood vessels, skin, muscles, such as those shown in FIG. 1A).

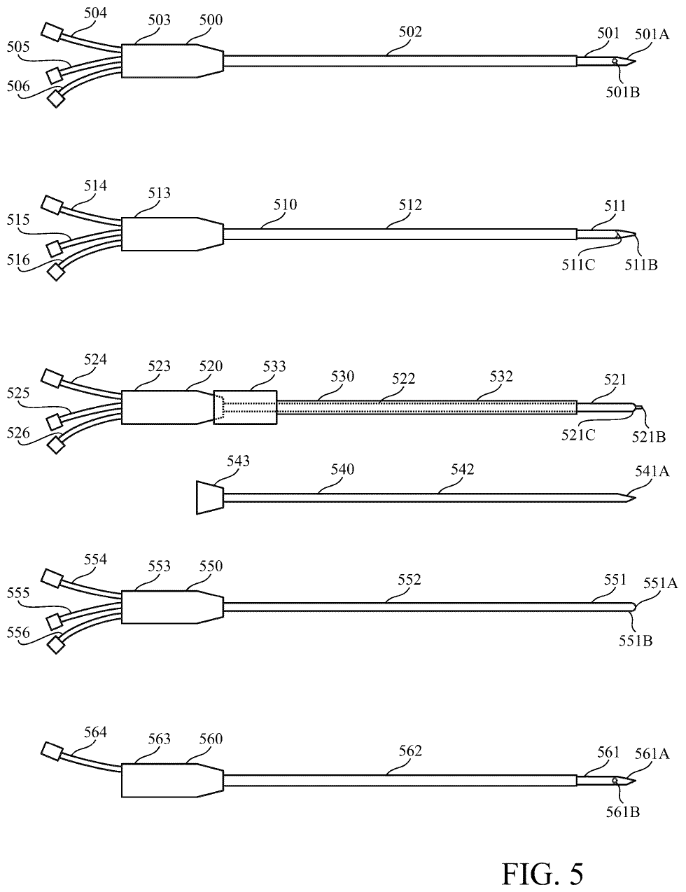

In some embodiments, a non-cooled electrode 150 can be an electrode that does not have a connection to a coolant source, such as a fluid pump 130; for example, such an electrode is electrode 560 in FIG. 5. In some embodiments, a non-cooled electrode 150 can be an electrode that has a connection to a coolant source, such as a fluid pump 130, but the coolant pump is not activated and coolant is not delivered to the electrode; such an electrode is electrode 150 in FIG. 1C.

It can be particularly advantageous to moving shot ablation in a region such as the thyroid gland that the ablation probe 150 provides for both cooled ablation (e.g. connection of the probe 150 to a cooling system, e.g. pump 130, and structures for circulation of coolant within the ablation probe shaft), and non-cooled ablation (e.g. a temperature sensor located within the active tip 151, i.e. the portion of the probe shaft around which tissue is ablated; temperature sensor 501B is located in active tip portion 501 of electrode 500); that the generator 100 includes control and user interface elements for both cooled ablation (e.g. power control 106D, impedance monitoring 104A, impedance plotting 102B, and impedance audio feedback 107AA, power monitoring 102B, power plotting 102A) and non-cooled ablation (e.g. a temperature controller 106C, temperature plotting 102C, and temperature audio feedback 107AA); and that the user interface of generator 100 include controls for easy switching between cooled and non-cooled ablation (for example, buttons 106E and 106EA in FIG. 1D, and common control of generator 100 and pump 130 output via connection 134 as shown in FIG. 1B, or via integration as shown in FIG. 1D); so that the user 180 can quickly adjust ablation size by cooling the ablation probe 150 (thereby enlarging the ablation size) or not cooling the ablation probe 150 (thereby reducing the ablation size).

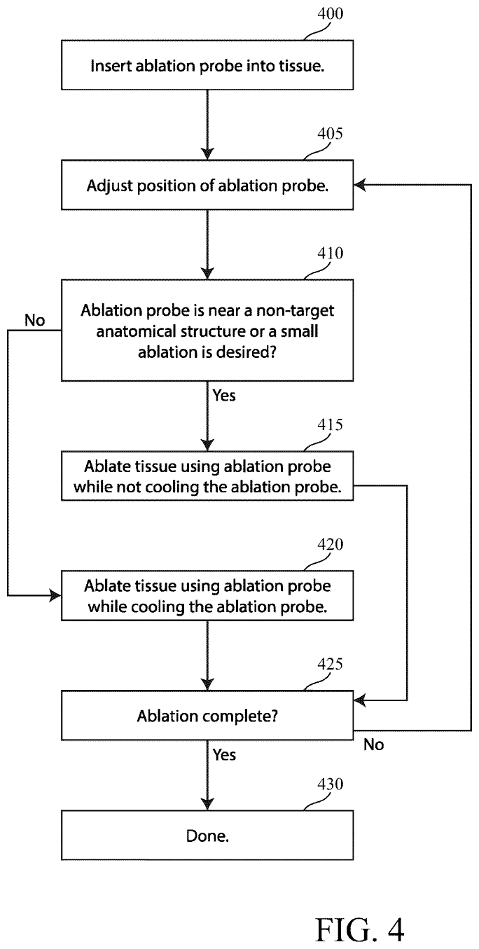

Referring now to FIG. 4, an embodiment of a method of cooled and non-cooled ablation is presented. In the first step 400, and ablation probe, such as probe 150, is inserted to into bodily tissue, such as the thyroid gland, uterus, or another organ. In the next step 405, the position of the ablation probe is adjusted to position desired for tissue ablation (e.g. a position in or near an anatomical structure of which ablation is desired), for example using image guidance (e.g. using an ultrasound machine 140) and/or using a nerve-stimulation method described in relation to FIGS. 2A, 2B, 2C, 2D, 3A, and 3B, or elsewhere herein. In the next step 410, the position of the ablation probe 150 is assessed for its proximity to non-target structures, i.e. structures of which ablation would be undesirable (such as nerves and other structures in and around the thyroid gland, see FIG. 1A), and/or the desired size of the ablation zone is assessed. If the ablation probe is near a non-target structure, or if the user desires a small ablation zone, then the next step is step 415 (i.e. the method follows the “Yes” arrow). On the other hand, if the ablation probe is not near a non-target structure, and the user desires a large ablation zone, then the next step is step 420 (i.e. the method follows the “No” arrow). In some embodiments of step 410, the assessment can exclude an assessment of non-target structures, so that only the desired size of the ablation zone is assessed. In some embodiments of step 410, the assessment can exclude an assessment of the desired size of the ablation zone per se, such that only proximity to a non-target structure is assessed. In step 415, non-cooled ablation of tissue is performed, i.e. the ablation probe is used to ablate tissue without cooling the ablation probe (as shown, for example, in relation to FIG. 1C). In step 420, cooled ablation of tissue performed, i.e. the ablation probe is used to ablate tissue while the ablation probe is cooled (as shown, for example, in relation to FIG. 1B). The next step after step 415 is step 425. The next step after step 420 is step 425. In step 425, the completeness of the ablation process is assessed. If the ablation is not complete, for example if there are other locations to be ablated in a moving shot ablation process, then the next step after step 425 is step 405 (i.e. the method follows the “No” arrow), and that step 405 and certain other steps are repeated as described above. However, if the ablation is complete, for example if all anatomical structures appear to have been destroyed by ablation, then the next step after step 425 is step 430 (i.e. the method follows the “Yes” arrow) and the method is complete. Such a method can have advantages for moving shot ablation and/or ablation in anatomical regions in which both target and non-target structures are in close proximity, such as the thyroid gland. Advantages of such a method include the ability to adjust the size, regularity, reproducibility, and speed of ablation by using or not using cooling of the ablation probe 150. In some embodiments, the method of FIG. 4 can be combined with the nerve-stimulation methods of FIGS. 3A and 3B, and other nerve-stimulation methods described herein. In particular, physiologic responses to nerve-stimulation can be used to select between cooled or non-cooled ablation. For example, there is an undesired response to nerve stimulation delivered via the ablation probe, such as voice change during a thyroid ablation procedure, at a low level of nerve stimulation, then non-cooled ablation can be selected to produce a smaller ablation zone, and thus, therapeutic destruction of tissue can be performed with lowered risk of adverse events (such as durable voice change). On the other hand, if there is an absence or low level of undesired response to nerve stimulation delivered via the ablation probe at a high level of nerve stimulation, the cooled ablation can be selected to produce a larger ablation zone, and thus, therapeutic destruction of tissue can be performed with greater speed or over a larger anatomic region with less concern about adverse events (such as durable voice change).

Referring now to FIG. 5, several embodiments of RF ablation electrodes 500, 510, 520, 550, 560 are set forth schematically; these electrodes are exemplary embodiments of the ablation probe 150. All such electrodes typically have a shaft that is predominantly rotationally symmetric about their long axes. An RF electrode, and an ablation probe in general, can have shaft length in the range 5 to 40 cm; shaft diameter in the range 23 to 10 gauge; and active tip length in the range 2 to 60 mm; however, in some embodiments, dimensions outside these ranges are possible. For thyroid ablation, useful dimensions for an RF electrode are: shaft length 7 to 17 cm; shaft diameter 17 or 18 gauge; active tip length 4 to 20 mm; however, dimensions outside these ranges are possible and can also be useful. Electrode 500 includes generator connection cable 504, coolant fluid inflow tube 505, coolant fluid outflow tube 506, proximal hub 503, and distal shaft, wherein the distal shaft comprises proximal electrically-insulated portion 502 and distal electrically-conductive active tip portion 501, the active tip 501 being the shaft portion from which an electrode-output signal (e.g. RF ablation signal and/or nerve-stimulation signal) produced by an RF generator, such as generator 100, is conducted to tissue. The distal tip point 501A of electrode 500 can be a sharp trocar point, a sharp flat bevel, conical point, a tissue-piecing point, or another type of medical needle point. When coolant is delivered to the electrode 500, for example by a pump 130, coolant flows from tube 505 along the shaft to a point the distal end 501A, and then back along the shaft to exit through tube 506, thereby cooling the active tip 501 of the electrode. In some embodiments, tip 501 can include an outflow port such that some of the coolant fluid, e.g. hypertonic saline, exits the electrode into the tissue; this can be referred to as perfusion ablation and can be used to enlarge the ablation zone. Inside the active tip 151 and substantially within or adjacent to the coolant flow path is temperature sensor 501B (drawn with a dotted outline), which provides for measurement of nearby tissue temperature and/or control of non-cooled ablation when the electrode 500 is not cooled, and for measurement of coolant fluid temperature when the electrode 500 is cooled.

In other embodiments of the present invention, a cooled-RF electrode includes a temperature sensor that extend beyond the portion of the electrode that is fluid cooled in order to estimate and control the tissue temperature elevation as a result of RF signal delivered through the electrode (e.g. electrodes 510 or 520). One advantage of these embodiments is that such electrodes can create an ablation zone that is larger and/or that formed in a faster manner, while also limiting the risk of tissue boiling and subsequent irregularity in the ablation zone that could cause inadvertent damage to tissue in or near the thyroid gland such as nerves, vessels, muscles, skin, trachea, or esophagus.

In some embodiments of ablation probes, such as electrode 510, temperature sensor is separated from the fluid flow path to more accurately measure tissue temperature near the electrode. Electrode 510 include generator connection 514, coolant inflow tube 515, coolant outflow tube 516, hub 513 at the electrode proximal end, and shaft comprising active tip 511 and electrically-insulated portion 512. The distal tip point 511B is sufficiently sharp to penetrate tissue and includes a temperature sensor. Barrier 511C (shown with dotted outline) within the active tip 511 prevents the flow of coolant fluid to the temperature sensor 511B and physically separates the coolant fluid path within shaft 511, 512 from the temperature sensor, such that the temperature measured by the sensor is more influenced by the surrounding tissue temperature, and is less influenced by the temperature of any coolant, than a temperature sensor (such as 501B in electrode 500) that resides in the coolant fluid flow path. An advantage of such a configuration is that generator 100 can measure, display, and control the temperature measured by sensor 511B while the electrode 510 is being cooled, thereby enlarging the ablation zone by means of electrode cooling, and also increasing the degree of control over tissue temperature relative to the degree of control when tissue temperature is not substantially measured and controlled. The configuration of electrode 510 has special application for moving shot ablation in anatomical regions that include many sensitive non-target structures, such as the thyroid gland, because its sharp tip point 511B provides for repeated movement of the electrode 510 to multiple locations, enlargement of the ablation zone by means of electrode cooling, and greater control over tissue temperature by means of the temperature sensor 511B that is physically separated from the electrode's coolant flow path.

Electrode 520 is another embodiment of an ablation probe in which the temperature sensor 521B is physically separated from the electrode's fluid flow path. Electrode 520 include generator connection 524, coolant inflow tube 525, coolant outflow tube 526, hub 523 at the electrode proximal end, and shaft comprising portions 521 and 522, both of which are electrically conductive. Electrode 520 is inserted via cannula 530, which includes hub 533 at its proximal end and electrically-insulated shaft 532 at its distal end. Shaft portion 522 is depicted with dotted outline within the shaft lumen of cannula 530. A distal portion of electrode hub 523 that engages within cannula hub 533 is also depicted with dotted outline. Active tip 521 is the portion of the shaft of electrode 520 that protrudes from the distal opening of the cannula shaft 532. The electrode tip 521 includes a distal extension 521B that includes a temperature sensor at its distal end. The coolant fluid path of electrode 520 ends at the primary end 521C of the shaft before the shaft outer diameter reduces to form extension 521B. As such, the temperature sensor 521B is physically and substantially thermally isolated from any fluid coolant within electrode 520. In some embodiments, cannula shaft 532 can be electrically uninsulated over a portion of its distal end, and thus, that distal portion forms a part of combined active tip with electrode shaft portion 521 by conductive contact between the inner surface of the lumen of cannula 530 and the outer surface of the electrode shaft 521, 522. In some embodiments, electrode 520 can include an electrically insulated proximal shaft portion, analogous to shaft insulation 512 of electrode 510, and the shaft 532 of cannula 530 can be either electrically insulated or electrically uninsulated. Cannula 530 can pierce bodily tissue by means of stylet 540, which is adapted be inserted into the lumen of cannula 530 when of electrode 520 is not so inserted. Stylet 540 includes a proximal hub 543, distal shaft 542, and a sharp distal point 541A. When cannula 530 and stylet 540 are unified by inserting stylet 540 into the lumen of cannula 530 such that their hubs 543 and 533 engage, the combination of cannula 530 and stylet 540 can be used to pierce tissue. The stylet 540 can then be replaced by an electrode, such as electrode 520 or 550, to form an ablation electrode and perform tissue ablation.

Electrode 550 is another embodiment of an RF electrode whose shaft 551, 552 does not include external electrical insulation, and can be inserted into the axial lumen of cannula 530 to form an ablation probe. Electrode 550 include generator connection 554, coolant inflow tube 555, coolant outflow tube 556, hub 553 at the electrode proximal end, and shaft comprising portions 551 and 552, both of which are electrically conductive. Electrode 550 is inserted via cannula 530, which includes hub 533 at its proximal end and electrically-insulated shaft 532 at its distal end. Active tip 551 is the portion of the shaft of electrode 550 that protrudes from the distal opening of the cannula shaft 532. The distal tip 551A of the shaft 551, 552 is rounded and includes a temperature sensor 551B. Temperature sensor 551B is positioned within the fluid flow path within shaft 551, 552.

Electrode 560 is an embodiment of an RF electrode that does not provide for cooling of the electrode active tip. Electrode 560 is analogous to electrode 500 except that it does not provide coolant tubing and coolant flow path within its shelf 561, 562. Electrode 560 include generator connection 564, hub 563 at the electrode proximal end, and shaft comprising distal active tip 561 and proximal electrically-insulated portion 562. The distal tip point 561A is sufficiently sharp to piece tissue and, in some embodiments, can be a trocar point or another tissue-piercing point used on medical needles. Active tip 561 includes a temperature sensor 561B (depicted using a dotted outline) whose measurement can be controlled by generator 100 for the purpose of non-cooled, temperature-controlled RF ablation. A strictly non-cooled electrode, such as electrode 560, has the advantage that its shaft 561, 562 can be thinner and less expensive than electrodes that provide for shaft cooling, because tubing and other structures for cooling within the shaft can be omitted. A thinner electrode can produce a smaller ablation zone and less tissue trauma due to insertion, which can have safety advantages, reduce procedure-related pain for the patient, and speed up post-ablation recovery. This can have special advantages when applied in sensitive anatomy, such as that in and around the thyroid gland.

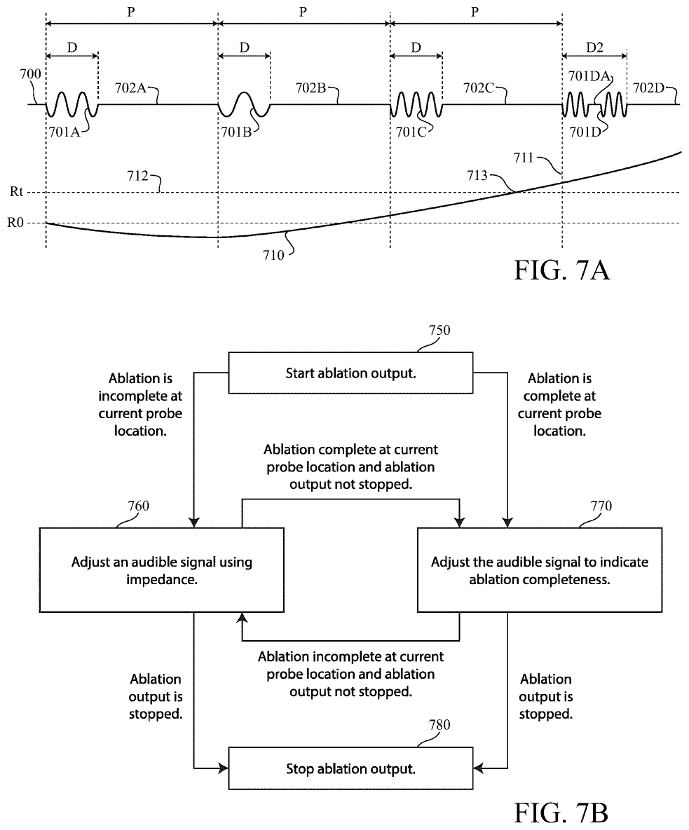

Referring now to FIGS. 7A and 7B, in another embodiment, the present invention relates to methods and systems that use audio signals (such as 107AA) to indicate the impedance (such as 104A) measured for an RFA signal delivered to (or near) the thyroid gland. In one embodiment, such a method includes the steps:
1 Convert the RF signal impedance to an audible signal, wherein the RF signal is delivered within thyroid gland.

In one example, the impedance 104A can determine the frequency of an audible tone 107AA. For example, the frequency of a tone can increase as the impedance increases (as shown in the example in FIG. 7A). In one example, the audible tone can take on only two configurations, one that indicates the impedance is below a threshold, and one that indicates the impedance is above a threshold. In another example, the audible signal can take on three configurations, each configuration indicating that the impedance falls within one of three impedance ranges. In another example, the audible signal can take on more than two configurations, each configuration indicating that the impedance falls within one of three or more impedance ranges. In another example, the audible signal can have a characteristic that varies substantially continuously with the impedance value. In one example, the impedance can determine one or more characteristics of the audible signal selected from the list: frequency, amplitude, pulse rate. In one example, the impedance can select among two or more words recorded as audible signals, e.g. “low impedance” or “high impedance” for lower and higher impedances respectively; “tissue heating” or “tissue boiling” for lower and higher impedances respectively; “tissue heating”, “impedance rising”, “move electrode” for lower, higher, and highest impedances respectively. In some embodiments, relative impedance values and/or impedance rises can be referenced to one or more of the following impedance values: the initial impedance value when the RF output was most recently turned on by the user, the impedance value when the RF output was initially turned on during the procedure, the most recent local minimum in the impedance time series, a selected impedance value. For example, the four carrier frequencies of beeps 701A, 701B, 701C, and 701D in audio signal 700 can represent four configurations of an audible signal 107AA; wherein the carrier frequency of tone 701A indicates that the measured impedance (plotted over time by graph 710) falls in a range that is near the initial measured impedance R0 (e.g. the range between R0−Rb and R0+Rb, where Rb is a numerical constant); wherein the lower carrier frequency of tone 701B indicates that the measured impedance falls in a range that is substantially lower than impedance R0 (e.g. the range of impedances that are less than or equal to R0−Rb); wherein the higher carrier frequency of tone 701C indicates that the measured impedance falls in a range that is substantially higher than impedance R0 but below threshold Rt (e.g. the range that is greater than or equal to R0+Rb, but less than Rt); wherein the highest carrier frequency of tone 701D indicates that the measured impedance falls the range that is greater than or equal to threshold Rt.

Referring now to FIG. 7A, a schematic example of a segment 700 of the waveform of an impedance-dependent audible tone 107AA is shown. The audio waveform 700 and an synchronous impedance graph 710 are plotted on the same horizontal time axis, and several vertical and horizontal dotted lines (such as 711 and 712) are included to show relative timing of theses time graphs 700, 710. The vertical value of signal 700 plots the amplitude of the audio signal 107AA. The vertical value of graph 710 plots the impedance value. The impedance value starts at initial value R0, decreases in value, then increases in value, ultimately exceeding impedance threshold Rt. Segment 700 of audio signal 107AA includes four pulses 701A, 701B, 701C, 701D that are delivered with period P. Each pulse is followed by a quiet period 702A, 702B, 702C, 702D; in particular, audio tone 701A is followed by quiet period 702A, audio tone 701B is followed by quiet period 702B, audio tone 701C is followed by quiet period 702C, audio tone 701D is followed by quiet period 702D. Pulses 701A, 701B, 702C are each pure sinusoidal waveforms, having duration D, and would sound to the user 180 like a single uninterrupted beep when played through a speaker. Pulse 701D comprises two pure sinusoidal waveforms, separated by a brief quiet period 701DA. Pulse 701D has total duration D2 and would sound to the user like a double beep when played through a speaker. In this example, duration D2 is longer than duration D. The frequency of the sinusoidal waveform during a pulse (referred to as the pulse's “carrier” or “fundamental” or “principal” frequency) varies with the impedance value 710 at the time of the pulse. Accordingly, the carrier frequency of the first pulse 701A corresponds to the initial impedance value R0. The carrier frequency of the second pulse 701B is lower than that of pulse 701A because the impedance is lower than R0 at the time of pulse 701B. The carrier frequency of the third pulse 701C is higher than that of pulse 701A because the impedance is higher than R0 at the time of pulse 701C. The carrier frequency of the fourth double pulse 701D is higher than those of pulses 701A, 701B, and 701C because the impedance is even higher at the time of pulse 701D. In addition, because the impedance at the time of pulse 701D is higher than threshold Rt, pulse 701D is specially distinguished as a double pulse to indicate an above-threshold impedance value to the user 180. In some embodiments, the pulses' carrier frequency F can be a linear function of the impedance value R within a certain range of impedance values, i.e. the F=a*R+b where a and b are constants. The period P can take a value is sufficiently short for the user 180 to timely respond to changes in impedance; preferably, the period P is in the range 0 to 1 seconds, where P=0 represents a carrier frequency that is continuously updated. A periodic impedance-based tone has an advantage that it also conveys temporal information to the user 180, without the user's having to look at the generator timer 104C. A period P of 1 second has the advantage the user can auditorily judge a duration in units of seconds simply by counting the number of pulses. In some embodiments, the pulse duration D can be fixed, and in some embodiments, the pulse duration D can vary. The pulse duration D can be in the range 0.05 to 0.5 seconds, in some embodiments. The pulse duration can be a value less than or equal to the pulse period P. The carrier frequencies of the impedance-based audio tone 107AA can be in the range audible to humans. The carrier frequencies of the audible signal 107AA can in the range 100 Hz to 3000 Hz; this has the advantage that the audible signal 107AA can serve both as an indicator of impedance and as an indicator that the electrosurgical output circuit of generator 100 is energized, as is required for electrosurgical equipment, such as RF generators, by IEC 60601-2-2:2017 section 201.12.4.2.101, thereby reducing potential interference between multiple audible signals and operator confusion. An audible signal 107AA having the features of signal 700, a pulse period P of 1 second, and pulse carrier frequencies in the range 100 to 3000 Hz, has several advantages, particularly for moving shot ablation and/or ablation in the thyroid gland: (a) it conveys rich information about variations in signal impedance that inform about an ablation process, (b) it clearly distinguishes impedance values that are above a threshold (for example a threshold that indicates tissue boiling or completion of an ablation process, (c) it conveys information about duration in units of seconds to the user, and (d) it can serve an indicator that the generator is delivering ablation output to the electrode.

In some embodiments, for example audio signal 700 in FIG. 7A, a characteristic of the audio signal 107AA varies with the measured impedance value, e.g. displayed in 104A, and, at times when the impedance is above a threshold value (e.g. impedance threshold Rt in FIG. 7A), the audio signal 107AA is adjusted to include a distinct feature such that a user 180 can clearly distinguish below-threshold tones (e.g. a single tone 701A, 701B, or 701C) from above-threshold tones (e.g. a double tone 701D). The threshold (e.g. Rt) can be an impedance value that is indicative of tissue boiling or impeding tissue boiling, which can be indicated by a high or rising impedance value. For example, the carrier frequency of the audio signal 107AA can vary monotonically with impedance, and above a threshold impedance value (e.g. Rt in FIG. 7A), an additional alert tone is added to the audio signal or, in another example, sounded instead of the sub-threshold audio signal. For example, the carrier frequency of a pulsed audio signal 107AA can vary monotonically with impedance, wherein for impedances below a threshold (e.g. Rt), the audio signal consists of a single audio pulse (e.g. pulses 701A, 701B, 701C); wherein for impedances above the threshold, the audio signal consists of two audio pulses (e.g. 701D). Such audio signals 107AA have an advantage of impedance-guided RF moving shot ablation methods (in which the electrode is typically cooled and the electrode power is controlled), particularly in the thyroid and uterus, because the varying carrier frequency indicates to the user the rate and direction of change in the measured signal impedance value and, thus, information about an evolving ablation process, and the distinct above-threshold feature (e.g. double pulse 701D, or longer duration pulse 701D) indicates to the user that tissue is boiling or near boiling and, thus, the that ablation process is at or nearing completion at the present electrode position, all without having to turn the user's attention away from the ultrasound display 141 to look at the display 101 of the RF generator 100. In some embodiments, the impedance threshold (e.g. Rt) for the audio signal 107AA can be replaced with a threshold for another parameter, wherein a value on one side of the threshold indicates completion, or near completion, of the ablation at the present probe 150 location, and a value on the other side of the threshold indicates that the ablation at the present probe 150 location is not yet complete or near complete. For example, the threshold can be a RF power threshold (such as Pt in FIG. 9A), wherein ablation completion is indicated by the RF power delivery being below a threshold (such as when the generator electrode output reaches a maximal voltage level and is thus unable to achieve a target power output level), and ablation non-completion is indicated by the RF power delivery being above the threshold.

One advantage of the embodiments of audible signal 107AA described in relation to FIGS. 7A and 7B is that the physician performing RFA in the thyroid under ultrasound guidance 140 does not need to look at the impedance reading 104A displayed by the generator 100 in order to monitor impedance while the physician's eyes are focused on looking at the ultrasound image 141 to guide the electrode 150 position and to assess tissue changes during RF energy delivery. Permitting the physician 180 to hold his/her visual attention on the ultrasound image used for electrode guidance has the advantage of reducing the likelihood of moving the electrode to an incorrect location and inadvertently damaging sensitive tissue in or near the thyroid, such as nerves, skin, muscle, trachea, blood vessels, esophagus.

In some embodiments, the audible signal 107AA can additionally and/or alternatively encode other characteristics of the RF signal, including, without limitation, the time since the RF output was most recently turned on, a duration of RF output, a voltage, a current, a power, an impedance, a time, the time elapsed since an impedance rise, the time elapsed since an impedance decrease. For example, an audible signal that encodes the duration since the RF output was turned on, and/or since the most recent drop in impedance that succeeds a rising in impedance (where the rise and the drop indicate, for example, tissue boiling near the electrode and movement of the electrode to tissue that is not boiling, respectively) has the advantage that a doctor performing ultrasound-guided RFA is informed, without having to look at the generator screen or being told by an assistance, about duration of tissue heating before an impedance rise that indicates tissue boiling (i.e. the duration of a “heating period”); this can facilitate moving shot ablation and the physician's decision making about whether to increase or decrease the intensity of the RFA output (e.g. the RF output power) in order to control the rate of tissue heating. In some embodiments, the audible signal can encode words that recite duration, e.g. “30 seconds” or “30 seconds have elapsed”. In some embodiments, the audible signal can indicate an elapsed duration at regular intervals, e.g. 1, 5, 10, 15, 20, 30, 60 second, or another temporal interval. Encoding of duration can be as spoken words, specialize tone, beeps, amplitude, frequency, or another audio characteristic or encoding.

In some embodiments, the RF signal can be applied to a uterine fibroid, rather than within the thyroid gland.

Referring now to FIG. 7B, a method using impedance-based audible signals 107AA is presented. The method starts in step 750 wherein RF ablation output is delivered by a generator 100 to an ablation probe 150 situated in target anatomy, such as a thyroid nodule or uterine tumor. Step 750 proceeds to step 770 if there is an indication that ablation is complete (or nearing completion) at the current position of electrode 150. An indication of ablation completeness can be that the RF signal impedance measured by the generator 100 is greater than a threshold value, such as Rt. An indication of ablation completeness can a drop in generator power output, for example below a threshold such as Pt in FIG. 9A. Otherwise, step 750 proceeds to step 760. In step 760, audible signal 107AA is adjusted depending on the RF signal impedance measured by generator 100. For example, the carrier frequency of the audio signal can be proportional to the impedance, such as for pulses 701A, 701B, and 701C in audio waveform segment 700. In step 770, audio signal 107AA is adjusted to indicate that ablation is complete (or nearly completion) at the present electrode position. For example, in embodiments where an above-threshold impedance is the indicator of ablation completeness, the carrier frequency of the audio signal 107AA can continue to increase with impedance. In some embodiments, the audible signal 107AA sounded during step 770 can be adjusted in amplitude, volume, or frequency; or it can be pulsed; or it can be pulses repeatedly; in order to distinctly convey completion (or near completion of ablation). In some embodiments, a distinct alarm signal can be sounded as audible signal 107AA during step 770. If the ablation output is stopped by the user 180, or a generator cut-off function is activated, during step 760 or step 770, then the next step is step 780. In step 780 RF ablation output from generator 100 to electrode 150 is halted. Otherwise, if ablation output is not stopped, step 760 transitions to step 770 if there is an indication that ablation is complete (or nearing completion) at the current position of electrode 150. And, if ablation output is not stopped, step 770 transitions to step 760 if the indication of ablation completeness or near completeness is no longer present, e.g. the impedance drops below the threshold value, e.g. Rt, which can occur if the user 180 move the electrode to heat another lotion in the target anatomy.¬ Embodiments of the method if FIG. 7B can be used to generate signal 700 of FIG. 7A. In some embodiment of step 770, the audible is signal is additionally adjusted using impedance.

In some embodiments where generator 100 is a microwave generator that delivers a microwave ablation signal to microwave antenna probe 150, audible signal 107AA can be varied according to the value and thresholds for a parameter that indicates the efficiency of microwave energy delivery to tissue.

In another embodiment, the present invention pertains to an RF generator 100 that is configured for moving-shot thyroid ablation, wherein the generator further includes a speaker, wherein the generator adjusts the audio signal 107AA delivered to the speaker based on a measured the RF signal impedance. For example, the frequency of a continuous or intermittent audible tone can increase as the RF signal impedance 104A increases. For example, the fundamental frequency of an intermittent or continuous audible beep can be a monotonic or strictly increasing function of the RF signal impedance 104A. For example, the frequency or number of beeps can change in response to RF signal impedance.

In one example, the audio signal 107AA varies such that it is presented in only two configurations, one that indicates the impedance is below a threshold, and one that indicated the impedance is above a threshold. In another example, the audio signal can take on at least three configurations, each configuration indicating that the impedance falls within one of at least three impedance ranges. In another example, the audible signal can have a characteristic (such as audio frequency) that varies substantially continuously with the impedance value. One advantage of the audio signal being able to take on more configurations, and, in particular, more than two configurations, is that increase and decreases of the impedance can be conveyed audibly with greater granularity to the physician performing moving shot RFA, thereby reducing the need for the physician to turn their visual attention away from the ultrasound image is the being used for electrode guidance and monitoring of ablation-related tissue changes. In some embodiments, the audio signal encodes spoken words that are indicative of the impedance value or configurations thereof.

In some embodiments, a characteristic of the audio signal 107AA represents an absolute impedance value. In some embodiments, a characteristic of the audio signal represents a relative impedance value, such as the difference between the present impedance and (1) the impedance measured at or before the instant the RFA output signal was turned on, either most recently (e.g. Z0 in FIG. 9A) or at the outset of the procedure; (2) the minimum impedance measured since the RFA output signal was activated; or (3) the minimum impedance measured since the last drop in impedance after a large increase in impedance (e.g. during heating period H3, the impedance at point 911), the large increase indicating tissue boiling or impeding boiling due to the RFA signal, the drop indicating that the electrode has moved to cooler tissue.

One advantage of this embodiment is that the physician 180 performing moving-shot RFA in the thyroid 193 under ultrasound guidance 140 does not need to look at the impedance reading 104A displayed by the generator 100 in order to monitor impedance 104A while the physician's eyes are focused on looking at the ultrasound image 141 to guide the electrode position and assess tissue changes during RF energy delivery. Helping the physician 180 to hold their visual attention on the ultrasound image 141 used for electrode guidance has the advantage of reducing the likelihood of moving the electrode to an incorrect location and inadvertently damaging sensitive tissue in or near the thyroid, such as nerves (15, 40), skin (191), muscle (192), trachea (50), blood vessels (10, 20), esophagus (45).

In some embodiments, the audio signal 107AA can additionally and/or alternatively encode other characteristics of the RF signal, including, without limitation, the time since the RF output was most recently turned on, a duration of RF output, a voltage, a current, a power, an impedance, a time, the time elapsed since an impedance rise, the time elapsed since an impedance decrease. For example, an audio signal that encodes the duration since the RF output was turned on, and/or since the most recent drop in impedance that succeeds a rising in impedance (where the rise and the drop indicate, for example, tissue boiling near the electrode and movement of the electrode to tissue that is not boiling, respectively) has the advantage that a doctor performing ultrasound-guided RFA is informed, without having to look at the generator screen or being told by an assistance, about duration of tissue heating before an impedance rise that indicates tissue boiling; this can facilitate moving shot ablation and the physician's decision making about whether to increase or decrease the intensity of the RFA output (e.g. the RF output power) in order to control the rate of tissue heating. In some embodiments, the audio signal can encode words that recite duration, e.g. “30 seconds” or “30 seconds have elapsed”. In some embodiments, the audio signal can indicate an elapsed duration at regular intervals, e.g. 1, 5, 10, 15, 20, 30, 60 second, or another temporal interval. Encoding of duration can be as spoken woods, specialize tone, beeps, amplitude, frequency, or another audio characteristic or encoding.

In some embodiments, the RF generator 100 can be additionally or alternatively be configured for moving-shot ablation of uterine fibroids.

In another embodiment, the present invention pertains to a system that includes an RF generator 100, an electrode 150, and an audio speaker, wherein the RF generator is configured to deliver an RF signal to the electrode and thereby ablate tissue that is in contact with the electrode; wherein the speaker is configured to generate an audio signal 107AA; wherein the system is configured to adjust a characteristic of the audio signal 107AA based on the impedance 104A of the RF signal; wherein the generator is configured to control the amplitude the RF signal (such as amplitude A in FIG. 2B); wherein the generator 100 is configured to prevent cut off the RF signal when the impedance of the signal's circuit rises. In some embodiments, such prevention of cut off can be omission of an open-circuit signal cut-off. In some embodiments, such prevention of cut off can be a limit on, or reduction of, the RF signal amplitude as the impedance increases, such that the impedance will tend not to exceed the generator's high-impedance cut-off threshold even with continued deliver of RF signal output. In some embodiments, the limit can be a limit of the maximum voltage of the RF signal. In some embodiments, the limit can be a limit on the power of the RF signal that decreases as impedance increases. In some embodiments, the amplitude of the RF signal can be a characteristic selected from the list: voltage, current, power. In some embodiments, the system can further include a fluid pump 130 that delivers fluid to the shaft of the electrode. In some embodiments, the system further includes a fluid pump 130 for fluid cooling of the electrode.

In some embodiments, the characteristic of the audio signal 107AA is its frequency. For example, the frequency of a continuous or intermittent audible tone can increase as the RF signal impedance increases. For example, the fundamental frequency of an intermittent or continuous audible beep can be a monotonic or strictly increasing function of the RF signal impedance. In some embodiments, the characteristic can be the audio volume, a beep repeat rate, a beep repeat count, a word (such as “low”, “medium”, or “high”) or another characteristic.

In one example, the characteristic of the audio signal 107AA is adjusted between only two configurations, one that indicates the impedance is below a threshold, and one that indicated the impedance is above a threshold. In another example, the audio signal characteristic is adjusted among at least three configurations, each configuration indicating that the impedance falls within one of at least three impedance ranges. In another example, the audio signal can have a characteristic (such as audio frequency) that varies substantially continuously with the impedance value. One advantage of the audio signal being able to take on more configurations, and, in particular, more than two configurations, is that increase and decreases of the impedance can be conveyed audibly with greater granularity to the physician performing moving shot RFA, thereby reducing the need for the physician to turn their visual attention away from the ultrasound image is the being used for electrode guidance and monitoring of ablation-related tissue changes.

In some embodiments, a characteristic of the audio signal represents an absolute impedance value. In some embodiments, a characteristic of the audio signal represents a relative impedance value, such as the difference between the present impedance and (1) the impedance measured at or before the instant the RFA output signal was turned on, either most recently or at the outset of the procedure; (2) the minimum impedance measured since the RFA output signal was activated; or (3) the minimum impedance measured since the last drop in impedance after a large increase in impedance, the large increase indicating tissue boiling or impeding boiling due to the RFA signal, the drop indicating that the electrode has moved to cooler tissue.

One advantage of this embodiment is that the physician performing moving-shot RFA in the thyroid, uterus, or another organ under ultrasound guidance does not need to look at the impedance reading displayed by the generator in order to monitor impedance while the physician eyes are focused on looking at the ultrasound image to guide the electrode position and to assess tissue changes during RF energy delivery. Helping the physician to hold their visual attention on the ultrasound image used for electrode guidance has the advantage of reducing the likelihood of moving the electrode to an incorrect location and inadvertently damaging sensitive tissue in or near the thyroid, such as nerves, skin, muscle, trachea, blood vessels, esophagus.

In some embodiments, the audio signal 107AA can additionally and/or alternatively encode other characteristics of the RF signal, including, without limitation, the time since the RF output was most recently turned on, a duration of RF output, a voltage, a current, a power, an impedance, a time, the time elapsed since an impedance rise, the time elapsed since an impedance decrease. For example, an audio signal that encodes the duration since the RF output was turned on, and/or since the most recent drop in impedance that succeeds a rising in impedance (where the rise and the drop indicate, for example, tissue boiling near the electrode and movement of the electrode to tissue that is not boiling, respectively) has the advantage that a doctor performing ultrasound-guided RFA is informed, without having to look at the generator screen or being told by an assistance, about duration of tissue heating before an impedance rise that indicates tissue boiling; this can facilitate moving shot ablation and the physician's decision making about whether to increase or decrease the intensity of the RFA output (e.g. the RF output power) in order to control the rate of tissue heating. In some embodiments, the audio signal can encode words that recite duration, e.g. “30 seconds” or “30 seconds have elapsed”. In some embodiments, the audio signal can indicate an elapsed duration at regular intervals, e.g. 1, 5, 10, 15, 20, 30, 60 second, or another temporal interval. Encoding of duration can be as spoken woods, specialize tone, beeps, amplitude, frequency, or another audio characteristic or encoding.

In some embodiments, the RF generator 100 can be additionally or alternatively be configured for moving-shot ablation of uterine fibroids.

Referring to FIGS. 1A, 1B, 1C, and 1D, in another embodiment, the present invention pertains to a system that includes an RF generator 100, a fluid pump 130, an RF electrode 150, an audio speaker, numerical display of generator readings (including impedance 104A, power 104B, temperature 104D, time 104C, energy 104G, current 104E, voltage 104D, and other RF generator readings), a graphical display of generator readings (for example, impedance 102B, power 102A, temperature 102C, energy current, voltage, and other RF generator readings) plotted on a time axis 102X, a foot switch 119, a data collection system. The RF generator 100 delivers RF output to the electrode 150. The pump 130 delivers cooling fluid to the electrode's active tip 151, from which RF current is delivered to tissue, thereby heating the tissue nearby the electrode.

The RF generator 100 and pump 130 are configured to coordinate starting and stopping of the RF generator output and pump's fluid delivery. In one mode of operation, when the user initiates output delivery with electrode cooling (“cooled RF”, such as shown in FIG. 1B), the generator and pump are coordinated to start the pump first, monitor the electrode temperature 104D, and only initiate the generator RF output once the electrode temperature 104D falls below a threshold, so that RF output only starts once the cooling fluid from the pump 130 has sufficiently cooled the RF electrode 150 (because it takes time for the cooling fluid, e.g. water or saline, to travel through tubing 155 from the pump 130 to the electrode 150, and because proper electrode cooling for “cooled RF” treatment ensures proper enhancement of tissue heating). In another mode of operation, when the user/physician initiates output delivery without cooling (“non-cooled RF”, such as shown in FIG. 1C), the generator output delivery started, but the pump 130 does not start. One advantage of the generator-pump coordination is at the generator 100 and pump 130 can be started or stopped by a single user action, simplifying thyroid RFA.

The footswitch 119 allows the user 180 to start and stop output delivery using the user's foot, as shown in FIG. 1D. Given the coordinated action of the generator 100 and pump 130, the footswitch 119 allows the generator 100 and the pump 130 to be operated in a single action of the footswitch 119. This is an advantage, because the physician 180 can start and stop RF output, including cooled RF output, simply with the physician's foot, while both hands are occupied with electrode 150 and ultrasound transducer 145 (as depicted in FIG. 1D), as is typically for thyroid RFA. In one example, the footswitch 119 initiates output delivery when the physician depresses the footswitch 119, and the footswitch stops output delivery, when the physician 180 releases the footswitch 119. One advantage of this type of footswitch 119 operation is that it is easy to understand what mode of the generator/pump being commanded by the user 180 using the footswitch 119, i.e. press down is on, release is off. Another advantage of this type of operation is that, because the user 180 must depress the footswitch 119 to continue RF output delivery, the user cannot lose track of the footswitch 119 during ablation, and thus can always promptly stop the output if needed, especially in the event of an emergency. This ease of understanding and simplicity of operation is an advantage during thyroid RFA where the physician and staff have many responsibilities to manage at the same time.

Another example of footswitch 119 operation is for the user to press and release (once, twice, or more times) to start output delivery, and the press and release again (once, twice, or more times) to stop the output delivery. Such a mode of operation can be used and has the advantage that additional generator/pump actions can be triggered by the user by the number and timing of footswitch presses; however, it does not have the same advantages of the mode of footswitch operation mentioned in the last paragraph.

In another example, the footswitch 119 can have more than one actuatable button (e.g. 119A, 119B, 119C), either housed in single device (as shown in FIG. 1B and FIG. 1C), or housed in separate devices. One advantage of such a multi-button footswitch (such as 119 in FIGS. 1B and 1C) is that the user 180 can initiate multiple types of generator 100 and pump 130 functions by actuation of a different footswitch button 119A, 119B, 119C, either using a single foot or using both feet. For example, one button 119A can start and stop the RF output, and one or more other buttons 119B, 119C can increase or decrease the RF output level. This can have special advantage for thyroid RFA because the physician's hands are occupied.

The numerical display of generator readings (104A-H, 104J, 103) can, in one example, be color-coded for easy identification and differentiation by the physician 180 and assistant(s). The numerical display of generator readings (104A-H, 104J, 103) can, in one example, be large and displayed upright so that they are visible from a distance. These features have the advantage of facilitating identification and viewing of generator readings from a distance and/or when the physician's visual attention must be split between the generator 100 and ultrasound 140 displays, such as during thyroid RFA. For thyroid RFA, observation of the impedance 104A with delivered RF energy is used to determine the completeness of ablation at a particular location and to decide when to ablate at another location.

The graphical display 102A, 102B, 102C of generator readings 104B, 104A, 104D over time has a special advantage during thyroid RFA because the graph 102A-C captures the recent history of those readings, which the physician 180 may miss because the physician 180 has to split the physician's visual attention between the generator 100 and the ultrasound image 141. Additionally, the physician 180 can use the graph 120A-C to assess not only the absolute value of generator readings, but also their timing and rate of change, which can be used to adjust technical aspects of the RF treatment, such as the generator output level 104B. For example, if the generator impedance 102B does not timely rise, or rises too slowly, the physician 180 can objectively ascertain the timing of the impedance rise, and decrease or increase the generator output level and thus slow or speed impedance rise of subsequent ablations, respectively. In thyroid RFA, observation of the impedance and, in particular, the timing and rate of increase in impedance, with delivered RF energy is used to determine the completeness of ablation at a particular location and to decide when to ablate at another location.

The audio speaker presents audible signals 107AA to the physician user that provide information about the generator readings. Such audible signals 107AA can provide information about any generator reading (104A-H, 104J, 103), including impedance, temperature, power, time, voltage, current, energy, as well as functions thereof, such as a reading's rate of change, changes in a reading, a reading exceeding or falling below a threshold, and other mathematical functions. Such audible signals 107AA can take the exemplary forms set forth throughout this text. For thyroid RFA and moving-shot RFA, the RF signal impedance is used to determine the completeness of ablation at a particular location and to decide when to ablate at another location. As such, audible signals 107AA related to impedance (such as beeps whose number or frequency indicates the impedance, or automatically generated speech stating the impedance value) have special advantage for thyroid RFA and moving shot RFA, particularly because the physician user's visual attention is split between the ultrasound image 141 and the impedance reading 104A.

A data collection system automatically saves generator 100 and pump 130 information (including generator readings (104A-H, 104J, 103) over time, computed summary statistics, user annotations, screen shots, and other data) to memory for later retrieval and analysis. This data collection system is preferably included in generator 100, but in some embodiments, it can be housed in a different structure. This has the advantage of reducing the burden on physician 180 and assistants to document procedure information, which has special significant for thyroid RFA and moving-shot RFA because of the diverse attentional and physical demands of such procedures. It also has the advantage that data from multiple past procedures can be analyzed collectively to determine trends and capture larger sample statistics across patients.

Taken together, the systems set forth in FIGS. 1A, 1B, 1C, 1D and in other figures and text herein have special advantages for conducting thyroid RFA and other RFA medical procedures in which the anatomical target is ablation by moving the electrode to create multiple ablations that conform to the size of an anatomical target using ultrasound guidance. The features of this system allow the doctor to keep doctor's visual attention on the ultrasound image 141 and to keep the doctor's hands on the RF electrode 150 and ultrasound transducer 145, by means of a numerical displays (104A-H, 104J, 103) that are color-coded and easy to identify and see from a distance; real-time graphical display of readings over time (102A, 102B, 102C) that allow the doctor to act on recent readings history even if the doctor's attention isn't constantly on the readings, but is rather on the ultrasound image 141 used for image guidance; real-time graphical information (102A, 102B, 102C; 160A-163A; 160B-163B) that allow the physician 180 to objectively ascertain the value, timing, and rate of change of impedance and other readings that are used for electrode guidance and control; audio feedback 107AA about impedance 104A and other generator readings (such as 104A-H, 104J, 103, 117A) that allow the doctor 180 to keep the doctor's attention on the ultrasound image 141 used for image guidance around sensitive functional structures in the neck (examples of which are shown in FIG. 1A); automatic synchronization between the generator 100 and pump 130 (e.g. via controls such as 106E and 106EA); foot-activated footswitch(es) 119 that allow for hand-free operation of RF output delivery, including stopping and starting, as well as adjustment of the RF output level (e.g. power); a data collection system that automatically stores generator and procedure information (including for the current procedure and for past procedures). These features of an ablation system improve the ease of performing an ablation procedure, reduces the need for the physician 180 to be assisted in performing the procedure, reduces the burden on assistants, reduces the physician's uncertainty in delivering destructive energy nearby sensitive functional anatomical structures (such as those shown in FIG. 1A), improves the physician's responsiveness to changing tissue conditions during ablation (including having timely information and timely ability to stop the RF output in the event of a problem), and speeds up a procedure that can require up to 30 minutes or more of constant attention and electrode manipulation by the physician 180.

In some more specific embodiments of the systems shown in described in relation to FIGS. 1A, 1B, 1C, and 1D, the RF generator 100 can additionally be configured to deliver a nerve stimulation signal (such as signal 200) to the electrode 150, either at the same time as the RF output signal is delivered (such as signal 250), at a different time than the RF output signal is delivery (such signal 200), or both depending on the mode of operation. This has an additional special advantage or thyroid RFA and other RFA treatments in which the anatomical target 195 is ablated by moving the electrode 150 to create multiple ablations 91, 92, 93, 94, 95, 96, 97, 98, 99 that conform to the size of an anatomical target 195 wherein the anatomical target 195 is nearby nerves 15, 40 of which damage can result in functional deficits and other adverse events, such as the recurrent laryngeal nerve 40 of which damage can result in permanent voice change during thyroid RFA.

Referring now to FIG. 8, in another embodiment, the present invention pertains to a system that includes an RF generator 100 configured to turn off its RF output automatically when the impedance 104A of the RF output signal increases by an amount (a “Relative Impedance Rise Threshold”; an example of this is the “relative impedance value” in step 805) relative to a past value of the impedance of the RF output signal (a “Reference Impedance”; an example of this is the “first impedance” in step 810). In one example, such an RF generator 100 turns off its RF output when it measures an impedance value 104A that is equal to, or that exceeds, the sum of the Reference Impedance and the Relative Impedance Rise Threshold (an example of this is step 820 in FIG. 8).

In some embodiments, the Relative Impedance Rise Threshold is a value selected from the list: a fixed value, a user-adjustable value, an absolute impedance value, a proportion of a past value of RF output signal impedance, a percentage of the Reference Impedance, the minimum of an absolute impedance value and a percentage of the Reference Impedance, a value that depends on the size of a metallic active tip of an RF electrode, 5 ohms, 10 ohms, 15 ohms, 20 ohms, 25 ohms, 30 ohms, 40 ohms, 50 ohms, 100 ohms, 200 ohms, a value in the range 1-3000 ohms.

In some embodiments, the Reference Impedance is a value selected from the list: the initial impedance value at the start of the procedure, the impedance value at or before the time at which the RF output is first turned on during a procedure, the impedance value at or before the most recent time at which the RF output was turned on (e.g. R0 in FIG. 7A, or Z0 in FIG. 9A), the minimum impedance value after the most recent time the RF output was turned on (e.g. impedance at point 910 in FIG. 9A), the minimum impedance value after the most recent instance of an impedance rise and subsequent fall (e.g. impedance at point 911 in FIG. 9A), the minimum impedance value after the most recent instance of an impedance rise and subsequent fall wherein the rise indicates tissue boiling around the electrode tip 151 and the fall indicates movement of the electrode to cooler tissue (e.g. impedance at point 910 in FIG. 9A).

One advantage of these embodiments of the present invention that involve a Relative Impedance Rise Threshold (for example the method of FIG. 8) is that the RF generator 100 can promptly turn off the RF output when its impedance rises by an amount that indicates tissue boiling due to the application of the RF output. When automated by the RF generator 100, such cut-off of the RF output can be executed more quickly than can executed by a human user 180, particularly when the RF output impedance 104A is rising quickly, as is typical of boiling around the active tip of an RF electrode 150 connected to an RF generator 100. Prompt discontinuation of RF output in response to boiling can reduce the amount of tissue charring around the active tip 151 of an RF electrode 150 to which the RF output is delivered. Such charring can have undesired effects, both medical and technical. For example, such charring may lead to prolonged recovery after ablation, increased procedure pain, and other undesired clinical effects. For example, such charring may increase the density of ablate tissue such that subsequent tissue penetration by the RF electrode 150 to which the RF output is applied is more difficult. For example, such charring can lead to sticking of tissue to the RF electrode 150 to which the RF output is applied, making movement of the electrode 150 more difficult. For example, delayed or missed high-impedance cut-off can prolong ablation procedures by continuing to apply RF output even after tissue boiling prevents effective heating of the tissue (because RF heating of tissue is less effective during boiling around the RF electrode tip 151). The impact of such undesired effects can be amplified for moving-shot RFA, such as in the thyroid or uterus, because RF output signal is applied at potentially numerous adjacent and/or overlapping locations in the tissue during the same procedure.

A high-impedance cut-off threshold that is relative to a past measured value (such as the “relative impedance value” in the method of FIG. 8) is advantageous over an absolute high-impedance cut-off threshold, because the former threshold can adapt to varying tissue impedance during an ablation procedure or due to use of various types or sizes of electrodes 150. This has special application during a moving-shot RFA procedures, such as thyroid RFA or intrauterine RFA, where RF output is delivered to an electrode 150 that moves to different parts of an organ 193 over the course of the procedure, and thus the tissue exposed to the RF output has varying type, density, temperature, and other characteristics that impact the initial tissue impedance when the output is turned on, because the tissue is inherently variable and/or because the tissue is changing over the course of the procedure. This also has special application to moving-shot RFA procedures, such as thyroid RFA, wherein the length of the active tip 151 of the RF electrode 150 to which the RF signal is applied is mechanically varied by the user over the course of the treatment, because the baseline impedance tends to vary with the length of the electrode active tip. Such electrodes have shaft insulation 502 that can slide in and out of the handle 503 to varying the length of the active tip 501. Because an absolute high-impedance cut-off threshold has varying difference relative to the initial (baseline) impedance, the promptness of high-impedance cut-off can vary over the course of moving-shot RFA procedures with variable-tip RF electrodes, or such automatic high-impedance cut-off can even fail, potentially leading to excessive, undesired tissue charring. In contrast, a relative high-impedance cut-off threshold can reliably and promptly turn-off the RF output even as the initial (baseline) impedance varies. By way of example, an example of a high-impedance cut-off threshold that is relative to a past measured value can be threshold Zt in FIG. 9A wherein Zt is set to Z0+c each time the RF output is turned on, where c is fixed constant and Z0 is updated as the initial impedance each time the RF output is turned. In such embodiments, the output signal would turn off at the end of heating period H1 when the impedance 902B reaches or exceeds Zt, rather than continuing after heating period H1, as depicted in FIG. 9A.

A system that includes high-impedance cut-off threshold can be combined with some or all of the features of the systems shown in, and described in relation to, FIGS. 1A, 1B, 1C, and 1D. Such a combined system can facilitate ultrasound-guided moving-shot ablation by allowing physically to focus on the ultrasound image 141, to avoid ineffective post-boil tissue heating, and to minimize tissue charring.

Referring now to FIG. 8, in another embodiment, the present invention pertains to a method having the following steps:
1. Turn on an RF ablation signal (step 800).
2. Determine a relative impedance value (step 805).
3. Measure a first impedance of the RF ablation signal at a first time (step 810).
4. Measure a second impedance of the RF ablation signal at a second time, wherein the second time is after the first time (step 815).
5. Turn off the RF ablation signal output if the second impedance is greater than or equal to the sum of the first impedance and the relative impedance value (step 820).

In some embodiments, if the conditions for turning off the RF ablation signal output are not satisfied in step 5 (also referred to as step 820), then the method can return to step 4 (also referred to as step 815) by following dotted arrow 825, and steps 4 and 5 (i.e. steps 815 and 820) can be repeated. The above method can be embodied in the RF generator 100 and thereby fully automated. The above method can be embodied in the RF generator 100 and thereby partially automated, for example, by providing a signal to a human user 180 indicating the conditions for turning off the RF output in step 4 above. The above method can be fully performed by a human 180. It is advantageous to fully automate the method of FIG. 8 in order to more rapidly perform the method and thereby to promptly responds to impedance changes, which can be fast in the case of tissue boiling in response to application of the RF signal. In some embodiments, the relative impedance value in step 805 can be a change in impedance that reliably and quickly indicates tissue boiling; for some electrodes and reference impedances (i.e. the first impedance in step 810), such a relative impedance value can be selected from the range 10-50 ohms. Referring to the impedance graph 710 in FIG. 7A, in one embodiment, the relative impedance value can be (Rt-R0); the first impedance can be R0; if the second impedance is any point on the graph 700 that is at or above the dotted line 712, then the RF ablation signal output turns off in step 820.

Referring now to FIG. 9A, in another embodiment, the present invention pertains to a system and method of using and reporting information about tissue heating and boiling during tissue ablation, particularly for moving-shot-type tissue ablation methods.

In some embodiments, an RF generator 100 can compute and report statistics on the duration of instances of tissue heating before a boiling (e.g. H1, H2, H3, or H4), i.e. the time between (A) either the instant of (1) RF turning on or (2) cessation of boiling (e.g. as indicated by a decrease in impedance from a higher range indicative of boiling to a lower range indicative of non-boiling, by the output power or current increasing to or toward its set value due impedance decreasing and/or output voltage dropping below a maximum limit, and/or by the ability of RF generator to continue heating tissue), and (B) the instant of boiling starting (e.g. as indicated by impedance rising above a threshold indicative of boiling, by the power or current output level dropping due to the generator reaching a maximum output voltage and impedance dropping, or by other indication of inability to continue effectively heating tissue). Such statistics can include, without limitation, a total, count, average, standard deviation, maximum, and minimum. For example, the average duration of tissue heating periods (e.g. (H1+H2+H3+H4)/4 is the average over the heating period durations H1, H2, H3, and H4 in FIG. 9A) can be reported. This has the advantage of indicating how aggressively the tissue is heated, the power level relative to the electrode tip size and the tissue characteristics, the average extent of tissue heating at each position, the relative proportions of direct ohmic heating and conductive heating, the likelihood and extent of increased tissue density due to heating, and the likelihood and extent for tissue charring. Such indications can be predictive of the extent of tissue shrinkage due to tissue heating, patient recovery period from ablation treatment, and inflammation or pain from ablation treatment. For example, the total duration of tissue heating can be reported (e.g. H1+H2+H3+H4 is the total duration of heating during period A1 in FIG. 9A); this may be less than the total duration for which RF energy is delivered (e.g. A1 in FIG. 9A), and can indicate the proportion of energy-delivery time for which the extent of ablated tissue was materially increasing (which is one example of the efficiency of the physician's ablation technique), the extent to which the total energy delivered is representative of actual increases in the extent of ablated tissue, and the proportion of the total procedure time was spent effectively expanding the ablation zone size rather than just locally boiling or charring tissue. For example, the count of instances of tissue heating before boiling can be reported (e.g. 4 is the count of instances of tissue heating H1, H2, H3, H4 in FIG. 9A); this has the advantage of characterizing the number of locations at which heating energy was delivered to the tissue, the number of times the electrode was moved to different locations in the target structure(s), and the size of individual ablation zones relative to the total extent of tissue ablated.

In some embodiments, an RF generator 100 can compute and report statistics on the duration of instances of tissue boiling, i.e. the time between (A) the instant of boiling starting (e.g. as indicated by impedance rising above a threshold indicative of boiling, the power or current output level dropping due to the generator reaching its maximum voltage and impedance dropping, or other indication of inability to continue effectively heating tissue), and (B) either the instant of (1) RF turning off or (2) cessation of boiling (e.g. as indicated by a decrease in impedance from a higher range indicative of boiling to a lower range indicative of non-boiling, the output power or current increasing to or toward its set value due impedance rising and/or requisite output voltage being lower than a maximum limit, and/or the ability of RF generator to continue heating tissue). Such statistics can include, without limitation, a total, count, average, standard deviation, maximum, and minimum. For example, the average duration of boiling periods can be reported (e.g. (B1+B2+B3)/4 is the average over the boiling period durations B1, B2, B3 in FIG. 9A). This has the advantage of indicating how aggressively the tissue is heated, the doctor or generator's responsiveness to indications of boiling (responses including either moving or electrode to a different unheated or partially heated location in the tissue, or stopping the RF output), the amount of power delivery that is not substantially effective at expanding the extent of heated tissue, the likelihood and extent of increased tissue density due to heating, and the likelihood and extent for tissue charring. For example, the total duration tissue boiling can be reported (e.g. B1+B2+B3 is the total duration of tissue boiling during period A1 in FIG. 9A); this may between zero and the total duration of RF energy delivery (e.g. A1 in FIG. 9A) and can indicate the portion of energy-delivery time during which the ablation zone size was not substantially increasing (which is an example of the efficiency of the physician's ablation technique). For example, the count of instances of tissue boiling can be reported (e.g. 3 is the count of boiling periods B1, B2, B3 in FIG. 9A); this has the advantage of characterizing the number of locations at which heating energy was delivered to the tissue, the number of times the electrode was moved to different locations in the target structure(s), and the size of individual ablation zones relative to the total extent of tissue ablated.

An example of ablation energy delivery is shown in FIG. 9A as a schematic graph of heating power 902A (drawn in a thicker line) and impedance 902B (drawn in a thinner line) plotted on the vertical axis 901 over time on the horizontal axis 900). In this example, power 902A is plotted in units Watts and impedance 902B is plotted in unit Ohms. Several horizontal and vertical dotted lines 912 are included to show correspondence between certain points on the axes 900, 901 and the graphs 902A, 902B. In this example, the ablation output (e.g. RF generator output) turns on at time 910, which is the beginning of time interval A1, and turns off at time 915, which is the end of time interval A1. The interval A1 comprises one instance of heating energy delivery. During this interval A1, the output power 902A ramps up to a set level (which can, in other examples, change over time, for example in response to user adjustment of the output-power setting value, as shown in the example graph 102A in FIG. 1B), and the impedance 902B decreases from its initial value as the tissue heats up. As tissue heating causes the tissue boiling and bubbles (e.g. 99 in FIG. 1A) to form around the tip of the ablation probe (e.g. the active tip 151 of an RF electrode 150), the impedance 902B rises. If the electrode tip 151 isn't moved out of this boiling region or the output stopped, the impedance rise will tend to increase to an extent that the RF output signal reaches a maximum voltage, and thus, the heating output power decreases (e.g. from a set power of 40 watts to a lower power of approximately 10 watts); this occurs during time intervals B1, B2, and B3 in FIG. 9A. Such energy delivery during boiling periods (such as B1, B2, B3) does not increase the ablation size quickly or materially, but rather heats tissue or fluid adjacent to the electrode and tends to increase tissue density and charring locally. Such boiling periods stop, and tissue heating continues, if the physician 180 moves the electrode active tip 151 to a tissue region such that the active tip 151 is not completely or substantially surrounded by boiling bubbles. Such tissue heating occurs in time intervals H1, H2, H3, and H4 in FIG. 9A. During interval B3, the physician 180 moved the probe 150 more quickly in response to impedance rise than during intervals B1 and B2, and as such the heating power decreased by a smaller extent (and, in some examples, such decrease may not occur at at) than it did in intervals B1 and B2. The physician 180 moved the electrode to unheated tissue quickly during the impedance rise labeled 903, and, as such, the impedance rise did not increase above the threshold Zt for detecting impedance rises that are indicative of substantial tissue boiling, and heating period H2 was not interrupted. In some examples, the threshold Zt can be a fixed impedance, an impedance relative to the initial impedance Z0 when the generator output is turned on, or an impedance relative to the most recent minimum impedance after either (A) the last time the output was turned on or (B) the last time tissue boiling stopped such that tissue heating could continue (e.g. as indicated by the impedance dropping from a range indicative of tissue boiling to a range indicative of insubstantial tissue boiling). In some example, Zt can vary during an instance of RF output delivery (such as A1) or across instances of RF output delivery (A1, A2, A3, . . . as described below). The total duration of energy delivery A1 comprises the heating periods H1, H2, H3, and H4, and the boiling periods B1, B2, and B3. The average heating time is the average of the durations of intervals H1, H2, H3, and H4. The total heating time is the sum of the durations of intervals H1, H2, H3, and H4. The portion of the treatment time during which the tissue was substantially heated is a quotient whose numerator is the sum of the durations H1, H2, H3, and H4, and whose denominator is the duration of interval A1. The number of heating intervals is the count of intervals H1, H2, H3, and H4, i.e. four. The average boiling time is the average of the durations of intervals B1, B2, and B3. The total boiling time is the sum of the durations of intervals B1, B2, and B3. The portion of the treatment time during which the tissue was substantially boiling is a quotient whose numerator is the sum of the durations B1, B2, and B3, and whose denominator is the duration of interval A1. The number of boiling intervals is the count of intervals B1, B2, and B3, i.e. three. Such averages, total, counts, and other statistics of heating and boiling periods can be computed over more than one period of energy delivery during a medical procedure; for example, there can be multiple instances of energy delivery with durations A1, A2, . . . , AN (where N is an integer that is the number of such instances of energy delivery) within which there are multiple instances of heating periods with durations H1, H2, . . . , HM (where M is an integer that is number of heating periods) and multiple instances of boiling periods with durations B1, B2, . . . , BP (where P is an integer that is the number of boiling periods). In such procedures, (H1+H2+ . . . +HM)/(A1+A2+ . . . +AN) is the portion of the procedure during which substantial tissue heating occurs, and (B1+B2+ . . . +BP)/(A1+A2+ . . . +AN) is the portion of the procedure during which tissue boiling, or insubstantial tissue heating, occurred. The total energy delivered during a procedure is the time integral of the power reading during energy delivery, and the average power is the total energy divided by the total energy delivery time (A1+A2+ . . . +AN). In some embodiments, the generator 100 can compute and/or report the total power delivery during heating periods, i.e. the time-integrated power PH during heating periods H1, H2, . . . , HM, and the average power delivered during such heating periods which is said time-integrated power PH divided by the total heating time (H1+H2+ . . . +HM); one advantage of such computations and report is that they can give an indication of the energy and power delivery that materially contributed to expansion of the extent of tissue ablated, excluding energy delivery that primarily sustained local boiling (i.e. the energy delivery during the boiling periods), and such indications can help the physician improve their technique and clinical outcomes due to ablations. In the embodiment of FIG. 9A, generator 100 defines heating periods as periods of time when ablation output of the RF generator is active and the impedance is below the threshold Zt, and generator 100 defines boiling periods as periods of time when ablation output is active and the impedance is above threshold Zt. In the example of FIG. 9A, Zt is the impedance value such that the generator output level is limited to be below a certain power level, and as such, when the impedance 902B rises above Zt, the power 902A level decreases. In other embodiments, Zt can be set to a different level that is indicative of boiling, impending boiling, or ineffective tissue heating. In other embodiments, a generator 100 can distinguish heating and boiling periods based directly a power threshold Pt, i.e. heating periods are time periods when the power level can exceed Pt, and boiling periods are time periods where the power level cannot exceed Pt (e.g. due to elevated tissue impedance). Such a threshold Pt is depicted in FIG. 9A, and in this example threshold Pt does not correspond directly to threshold Zt; that is, use of the illustrated Zt and Pt would not lead to the same assessment of heating and boiling periods (for example, use of threshold Pt would not detect boiling period B3 because power dip 904 does not fall below threshold Pt). In other examples, Zt and Pt could be equivalent. It is understood that thresholds used to distinguish heating and boiling periods can be set to different values depending on the model of tissue heating and boiling, tolerance for false positive and false negative detections of heating or boiling, and other factors. In some embodiments, the impedance threshold Zt (or, in some embodiments, power threshold Pt) can be used to trigger the use of a distinct feature in audio signal 107AA that indicates to the user 180 the presence of a boiling period, as described, for example, in relation to FIGS. 7A and 7B.

Referring now to FIG. 9B, a method of characterizing the heating and non-heating periods of an ablation process is presented. Non-heating periods include periods when energy is not being delivered (such as stoppages or pauses of the ablation output in between instances of ablation output delivery) and period when tissue around the ablation probe has caused substantially boiling such that tissue heating is ineffective. This method can be applied to stored data from an ablation procedure, or it can be applied to data from an ablation procedure as it is collected in real time. The method starts in step 950 wherein time series of generator readings from RF ablation process, such as impedance and power measurements during a moving shot ablation process, are processed at each time point in the time series. Step 950 proceeds to step 970 if the time-series data points currently being processed indicate that ablation is ineffective (e.g. tissue boiling, high impedance, above-threshold impedance, low power output, below-threshold power output, an indication of ablation completeness or near completeness at the current position of the ablation probe 150). An indication of ablation ineffectiveness can be that the RF signal impedance measured by the generator 100 is greater than a threshold value, such as Zt. An indication of ablation ineffectiveness can be a drop in generator power output below a threshold, such as Pt. Otherwise, step 950 proceeds to step 960. In step 960, the current data point(s) of the ablation time series are labeled as being part of a heating period, i.e. a portion of the ablation process during which ablation energy is effectively increasing the size of the heated ablation zone. In step 970, the current data point(s) of the ablation time series are labeled as being part of a non-heating period, i.e. a period during which little to no energy is applied or during which applied energy is not likely to substantially expand the size of the ablation zone. If all ablation data has been segmented as being “heating” or “non-heating” step 960 or step 970, then the next step is step 980. In step 980 the data segmentation is used to compute statistics for the data during heating periods and during non-heating periods, the statistics being of the same type as those described for heating and boiling periods in relation to FIG. 9A. Otherwise, if processing of all data of the ablation process is yet not finished, then step 960 transitions to step 970 if the next data point to be labeled indicate that heating is ineffective or the ablation output is not active. And, if processing of all data of the ablation process is yet not finished, then step 970 transitions to step 960 if the next data points to be labeled indicate that ablation heating is effective, e.g. the impedance is below the threshold value Zt, or the power is above a threshold value Pt, which can occur if the user 180 recently moved the electrode to heat a new lotion in the target anatomy.¬ In some embodiments, the method of FIG. 9B can be used to characterize heating and boiling periods of an ablation process, excluding periods when the RF ablation output is inactive; in such embodiments, “non-heating periods” are limited to “boiling periods”. In embodiments where a generator 100 and ablation probe 150 ablate tissue by cooling the tissue, such as cryoablation, the method of FIG. 9B can be used by replacing “heating” with “cooling”.

In some embodiments, as described herein, the generator 100 output for heating-energy delivery can be a radiofrequency (RF) signal. In some embodiments, the generator 100 output for heating-energy delivery can be a microwave signal. In some embodiments, the generator 100 output for heating-energy delivery can be another type of electrical, acoustic, or other type of energetic signal. In some embodiments, the impedance 104A can be measured using a radiofrequency signal, and in some examples, that impedance-measurement radiofrequency signal can be the same radiofrequency signal as that used for heating-energy delivery. In some examples, the impedance measurement signal can be another electrical signal, such as a DC signal, low-frequency signal, a 50 Hz signal, and signal with frequency less than then radiofrequency range, or another type of electrical signal. In some embodiments, a generator 100 that heats tissue using a microwave signal can include a second impedance-measurement signal delivered from a conductive contact on the microwave antenna probe 150 (for example located at or near the distal end 151 of the probe when microwave ablation energy is principally delivered to tissue); one advantage of such a system is that impedance readings 104A can provide information about the tissue-heating process due to microwave energy delivery, even in the absence of tissue-temperature measurement, for example, due to the microwave antenna and indwelling temperature sensor being cooled during microwave energy delivery. In some embodiments, a first electrical signal can be used for heating (e.g. radiofrequency, microwave, IRE, DC, acoustic energy generator), and a second electrical signal can be used for impedance measurement.

Throughout this disclosure, “fig.” and “figure” are have the same meaning, irrespective of capitalization.

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

What is claimed is:

1. A system for radiofrequency tissue ablation, the system including a radiofrequency generator, a radiofrequency electrode, and a coolant pump; wherein the generator delivers a radiofrequency ablation signal the electrode, the radiofrequency ablation signal being adapted to ablate a tissue via the electrode; the generator is adapted to measure a power and an impedance of a radiofrequency ablation signal; the generator displays a graph of the measured impedance over time and a graph of the measured power over time; the generator produces an audible tone that indicates the value of the measured impedance; activation of the pump cools the electrode; deactivation of the pump stops cooling of the electrode; the system includes a cooled-RF mode of operation in which the generator delivers a radiofrequency ablation signal to the electrode and the pump cools the electrode.

2. The system of claim 1, wherein the electrode further includes a temperature sensor, and wherein the generator measures the electrode temperature.

3. The system of claim 2 wherein the system includes a non-cooled mode of operation in which the generator delivers a radiofrequency ablation signal to the electrode, the pump does not cool the electrode, and the generator controls the electrode temperature.

4. The system of claim 2 wherein, in the cooled-RF mode of operation, the generator delivers the radiofrequency ablation signal only if the electrode temperature is below a cooling temperature threshold.

5. The system of claim 2 wherein the generator displays a graph of the measured electrode temperature.

6. The system of claim 1 wherein the generator is adapted to activate and deactivate the pump.

7. The system of claim 1 wherein, in the cooled-RF mode of operation, the generator regulates the measured power of the radiofrequency ablation signal.

8. The system of claim 1 wherein, if the measured impedance is below an impedance-audio threshold, the generator configures the audible signal to be in a first form wherein a characteristic of the audible signal changes with changes in the measured impedance; and wherein, if the measured impedance is above the impedance-audio threshold, the generator configures the audible signal to be in a second form that is audibly distinct from the first form.

9. The system of claim 1 wherein the system includes a nerve-stimulation mode of operation in which the generator delivers a nerve-stimulation signal to the electrode.

10. The system of claim 1 wherein the generator includes a cut-off control wherein, in modes of operations in which the generator delivers a radiofrequency ablation signal to the electrode, the generator automatically stops delivery of the radiofrequency ablation signal if the measured impedance rises above a relative impedance threshold, wherein the relative impedance threshold is adapted to be higher than a prior measured impedance value.

11. The system of claim 10 wherein the prior measured impedance value is the measured impedance at the time when delivery of the radiofrequency ablation signal was last initiated.

12. The system of claim 1 wherein the audible signal is adapted to take at least three forms, wherein each form indicates that the measured impedance is in a different range of impedance values.

13. The system of claim 1 wherein the graphs of impedance and power are plotted on the same time axis.

14. The system of claim 1 wherein the generator is adapted to measure an energy of the radiofrequency ablation signal.

15. The system of claim 1 wherein the generator is adapted to measure a total energy of the radiofrequency ablation signal over more than one instance of delivery of the radiofrequency ablation signal.

16. The system of claim 1 wherein in the generator is adapted to distinguish measurements collected during heating periods from measurements collected during non-heating periods, heating periods being periods of time in which measurements indicate that delivery of the radiofrequency ablation signal to the electrode is materially increasing the size of an ablation zone in a tissue, non-heating periods being period of time in which the measurements indicate that delivering of the radiofrequency ablation signal to the electrode is not materially increasing the size of the ablation zone in the tissue; wherein the generator is adapted to display or record statistics about the value and timing of measurements in the heating periods; and wherein the generator is adapted to display or record statistics about the value and timing of the measurements in the non-heating periods.

17. The system of claim 1 wherein the system is adapted for moving-shot radiofrequency tissue ablation in an organ selected from the list: thyroid, uterus, kidney, liver, lung, and an organ in the human body.

18. The system of claim 1 and further including a footswitch, wherein the footswitch is adapted to activate and deactivate delivery of the radiofrequency ablation signal to the electrode.

19. The system of claim 18 wherein the footswitch is adapted to adjust a target parameter for delivery of radiofrequency ablation selected from the list: power, temperature, voltage, current, a parameter of the radiofrequency ablation signal.

20. The system of claim 1 wherein the generator includes a data storage system that stores the measurements.
